(19) United States
(12) Patent Application Publication
(10) Pub. No.: US 2016/0201031 A1
(43) Pub. Date: Jul. 14, 2016

(54) COMPOSITION AND METHODS FOR CULTURING CELLS

(71) Applicant: THE REGENTS OF THE UNIVERSITY OF CALIFORNIA, Oakland, CA (US)

(72) Inventors: George A. Brooks, Berkeley, CA (US); Rajaa Hussien, Berkeley, CA (US); Bertrand Rihn, Berkeley, CA (US)

(21) Appl. No.: 14/912,384

(22) PCT Filed: Aug. 20, 2014
(86) PCT No.: PCT/US14/51899

§ 371 Date: Feb. 16, 2016

Related U.S. Application Data
(60) Provisional application No. 61/868,430, filed on Aug. 21, 2013.

(51) Int. Cl. C12N 5/079; G01N 33/68; C12N 5/09

(52) U.S. Cl. CPC C12N 5/0618; C12N 5/0693; G01N 33/6848; G01N 2560/00; G01N 2570/00; C12N 2501/40; C12N 2501/10; C12N 2500/84; Y10S 977/773; Y10S 977/923; B82Y 5/00

(57) ABSTRACT

The present disclosure provides compositions for in vitro culture of cells. The present disclosure provides methods for in vitro culture of cells. The present disclosure provides methods for extracting proteins from a serum sample.

### CROSS-REFERENCE

This application claims the benefit of U.S. Provisional Patent Application No. 61/868,430, filed Aug. 21, 2013, which application is incorporated herein by reference in its entirety.

### INTRODUCTION

The size, penetrative, carrier, and other properties of nanoparticles (NPs) allow them to enter the human body through inhalation, digestion, injection, and skin contact. Within the body, NPs can be transported to organs, tissues and cells far from the site of exposure, where NPs cross anatomical barriers and penetrate live cells and their organelles. EUDRAGIT® RS nanoparticles are a non-biodegradable, positively charged copolymer.

### Literature

Eidi et al. (2010) Int. J. Pharm. 396:156; Dekeyser et al. (2007) J. Biomed. Mater. Res. A. 87:116; Lamprecht et al 2006 Nanotechnology 17 (June), pg. 3673; Yoo et al, Int J Pharm. 2011 Jan. 17;403(1-2):262-7; Eidi, Int J Pharm. 2012 Jan. 17;422(1-2):495-503; Pignatello et al, Eur J Pharm Sci. 2002 July;16(1-2):53-61; Bhardwaj et al, Acta Pol Pharm. 2010 May-Jun;67(3):291-8; Schaffazick et al, Eur J Pharm Biopharm. 2008 May;69(1):64-71; Gargouri et al, Technol Cancer Res Treat. 2009 December;8(6):433-44; Jiao et al, Circulation. 2002 Jan. 15;105(2):230-5; Wörle-Knirsch et al., Nano Lett. 2006 June;6(6):1261-8.

### SUMMARY

The present disclosure provides compositions and methods for in vitro culture of cells and for extracting proteins from a serum sample.

### BRIEF DESCRIPTION OF THE DRAWINGS

FIGS. 1A-F depict the effect on metabolic activity of cells following exposure to various amounts of EUDRAGIT® nanoparticles (ENPs).

FIGS. 2A-E depict the effects of exposing cells to various amounts of ENPs on cell proliferation, total protein, and cell count in HMEC 184 cells.

FIG. 3 depicts HMEC 184 cells after 3-day exposure to Nile red-ENP (25 μg/ml) as observed with a confocal microscope.

FIGS. 4A-B depict the effects of exposing cells for 3 days to 25 μg/ml ENPs on mitochondrial volume and protein content.

FIGS. 5A-B depict metabolic activity of HMEC 184 cells following 48 h exposure to ENPs.

FIGS. 6A-C depict metabolic activity, cell proliferation, microscopic analysis of neural progenitor cells following 24 h exposure to various amounts of ENPs.

FIG. 7 depicts linear regression analysis between the logarithm of plasma concentration (nmol) and the logarithm of relative abundance as obtained by mass spectrometry data.

FIG. 8 presents a table of commonly shared domains of proteins purified by ENPs.

FIGS. 9A-I present a table of proteins from FBS that were purified by contacting the FBS with ENPs. The proteins were identified with MALDI-TOF mass spectrometry.

FIG. 10 presents a table that presents relative abundance at the domain level.

FIGS. 11A-F present a table that lists significantly upregulated genes in HMEC 184 cells following 24 h exposure to ENPs.

FIGS. 12A-B present a table depicting functional annotation analysis of microarray data sets.

### DEFINITIONS

The term “growth factor” as used herein refers to a substance capable of stimulating cellular growth. The term “cytokine” as used herein refers to growth factors related to the immune system. As such, the term “growth factor” encompasses the term “cytokine”. Exemplary growth factors include, but are not limited to: Adrenomedullin (AM), Angiopoietin (Ang), Autocrine motility factor, Bone morphogenetic proteins (BMPs, e.g., BMPs 1-8, BMP10, BMP15, and the like), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha(TGF-α), Transforming growth factor beta(TGF-β), Tumor necrosis factor-alpha(TNF-α), Vascular endothelial growth factor (VEGF), Wnt Signaling Proteins (e.g., Wnt3a), placental growth factor (P1GF), ILs(Interleukins) (e.g., IL-1, IL-2, IL-3, IL-4, IL-5, IL-6, IL-7, and the like).

“EUDRAGIT®” is the brand name for a diverse range of polymethacrylate-based copolymers. It includes anionic, cationic, and neutral copolymers based on methacrylic acid and methacrylic/acrylic esters or their derivatives.

“EUDRAGIT® RS” is a EUDRAGIT® polymer comprising ethyl acrylate, methyl methacrylate and a low content of methacrylic acid ester with quaternary ammonium groups. The CAS number of EUDRAGIT® RS is 33434-24-1. The Chemical/IUPAC name of EUDRAGIT® RS is Poly(ethyl acrylate-co-methyl methacrylate-co-trimethylammonioethyl methacrylate chloride) 1:2:0.1.

The term “naked nanoparticles” as used herein refers to unloaded nanoparticles. As is known by one of ordinary skill in the art, nanoparticles can be contacted with a variety of compounds in order to “load” the nanoparticles. Loaded nanoparticles can then be used to deliver the cargo (i.e., the compounds loaded onto the nanoparticles) to, for example, a cell by contacting the cell with loaded nanoparticles. Naked nanoparticles are nanoparticles that have not been loaded prior to use in a subject method.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a culture medium” includes a plurality of such culture media and reference to “the epithelial cell” includes reference to one or more epithelial cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides compositions for in vitro culture of cells. The present disclosure provides methods for in vitro culture of cells.

### Cell Culture Compositions

The present disclosure provides compositions for in vitro culture of cells (e.g., which can be used in methods of culturing a call, methods of increasing cellular metabolism, methods of increasing metabolic activity of a cell, methods of increasing cellular growth, and the like). A cell culture composition of the present disclosure comprises a EUDRAGIT® RS nanoparticle and one or more components of standard culture media.

EUDRAGIT® nanoparticles suitable for use in a subject cell culture composition can be used at a final concentration (e.g., concentration after dilution into culture media) range of from 2 μg/ml to 300 μg/ml (e.g, from 2 μg/ml to 300 μg/ml, from 2 μg/ml to 250 pg/ml, from 2 μg/ml to 200 μg/ml, from 2 μg/ml to 150 μg/ml, from 2 μg/ml to 100 pg/ml, from 2 μg/ml to 75 μg/ml, from 2 μg/ml to 50 μg/ml, from 2 μg/ml to 40 μg/ml, from 2 μg/ml to 35 μg/ml, from 2 μg/ml to 30 μg/ml, from 2 μg/ml to 28 μg/ml, from 2 μg/ml to 25 μg/ml, from 5 μg/ml to 300 μg/ml, from 5 μg/ml to 250 μg/ml, from 5 μg/ml to 200 μg/ml, from 5 μg/ml to 150 μg/ml, from 5 μg/ml to 100 μg/ml, from 5 μg/ml to 75 pg/ml, from 5 μg/ml to 50 μg/ml, from 5 μg/ml to 40 μg/ml, from 5 μg/ml to 35 μg/ml, from 5 μg/ml to 30 μg/ml, from 5 μg/ml to 28 μg/ml, from 5 μg/ml to 25 μg/ml, about 10 μg/ml to 300 μg/ml, from 10 μg/ml to 250 μg/ml, from 10 μg/ml to 200 μg/ml, from 10 μg/ml to 150 μg/ml, from 10 μg/ml to 100 μg/ml, from 10 μg/ml to 75 μg/ml, from 10 μg/ml to 50 μg/ml, from 10 μg/ml to 40 μg/ml, from 10 μg/ml to 35 μg/ml, from 10 μg/ml to 30 μg/ml, from 10 μg/ml to 28 μg/ml, from 10 μg/ml to 25 μg/ml, about 15 μg/ml to 300 μg/ml, from 15 μg/ml to 250 μg/ml, from 15 μg/ml to 200 μg/ml, from 15 μg/ml to 150 μg/ml, from 15 μg/ml to 100 μg/ml, from 15 μg/ml to 75 μg/ml, from 15 μg/ml to 50 μg/ml, from 15 μg/ml to 40 μg/ml, from 15 μg/ml to 35 μg/ml, from 15 μg/ml to 30 μg/ml, from 15 μg/ml to 28 μg/ml, from 15 μg/ml to 25 μg/ml, from 22 μg/ml to 27 μg/ml, from 20 μg/ml to 30 μg/ml, about 15 μg/ml, about 17 μg/ml, about 20 μg/ml, about 22 μg/ml, about 25 μg/ml, about 27 μg/ml, or about 30 μg/ml).

A cell culture composition of the present disclosure provides for increased cell viability and/or metabolic activity of cells cultured in vitro in such cell culture composition, compared to the viability and/or metabolic activity of the cells grown in the culture composition in the absence of the ENPs.

### Metabolic Activity

In some cases, a cell culture composition of the present disclosure provides for an at least 10%, at least 20%, at least 25%, at least 50%, at least 75%, at least 2-fold, at least 5-fold, or at least 10-fold, or more than 10-fold, increased metabolic activity of a cell (i.e., increased cellular metabolism), e.g., relative to the metabolic activity of the cell prior to contact with a subject cell culture composition. In some cases, such a change can be measured, for example, relative to a control cell (e.g., a cell cultured in the absence of a subject cell culture composition (e.g., cultured in the absence of ENPs)).

Methods to measure metabolic activity (e.g., metabolic activity of a cell) will be known to one of ordinary skill in the art and any convenient method can be used. One non-limiting example of a suitable method to measure metabolic activity is a tetrazolium dye assay. A tetrazolium dye assay measures cellular metabolic activity via NAD(P)H-dependent cellular oxidoreductase enzymes (indicators of metabolically active mitochondria). Examples of suitable tetrazolium dyes for use in a tetrazolium dye assay include, but are not limited to, an MTT, an XTT, an MTS, and a WST.

MTT (3-(4,5-dimethylthiazol-2-yl)-2,5-diphenyltetrazolium bromide, a yellow tetrazole), is reduced to purple formazan in living cells. A solubilization solution (e.g., dimethyl sulfoxide, an acidified ethanol solution, or a solution of the detergent sodium dodecyl sulfate in diluted hydrochloric acid) is added to dissolve the insoluble purple formazan product into a colored solution. The absorbance of this colored solution can be quantified by measurement at a certain wavelength (usually between 500 and 600 nm) using a spectrophotometer. The absorption maximum is dependent on the solvent employed.

XTT (2,3-bis-(2-methoxy-4-nitro-5-sulfophenyl)-2H-tetrazolium-5-carboxanilide) has been proposed as a substitute for MTT, yielding higher sensitivity and a higher dynamic range. The formed formazan dye is water soluble, avoiding a final solubilization step.

MTS (3-(4,5-dimethylthiazol-2-yl)-5-(3-carboxymethoxyphenyl)-2-(4-sulfophenyl)-2H-tetrazolium), in the presence of phenazine methosulfate (PMS), produces a formazan product that has an absorbance maximum at 490-500 nm in phosphate-buffered saline.

WSTs (Water soluble Tetrazolium salts) are a series of water soluble dyes similar to, but different than MTT. WSTs were developed by introducing positive or negative charges and hydroxy groups to the phenyl ring of tetrazolium salt. Assays, developed to give different absorption spectra of the formed formazans. WST-1 (2-(4-Iodophenyl)-3-(4-nitrophenyl)-5-(2,4-disulfophenyl)-2H-tetrazolium) and WST-8 (2-(2-methoxy-4-nitrophenyl)-3-(4-nitrophenyl)-5-(2,4-disulfophenyl)-2H-tetrazolium), for example, can be considered advantageous in that they are reduced outside of cells, and yield a water-soluble formazan directly (such assays do not require a solubilization step). Because WSTs are reduced outside of cells, the formazan product forms extracellularly instead of intracellularly, and the assay therefore has reduced cell toxicity.

In some embodiments, a subject method comprises measuring the metabolic activity of a cell using a tetrazolium dye other than MTT (3-(4,5-dimethylthiazol-2-yl)-2,5-diphenyltetrazolium bromide, a yellow tetrazole). In some embodiments, a subject method comprises measuring the metabolic activity of a cell using a tetrazolium dye selected from the group consisting of: XTT, MTS, WST-1, and WST-8.

Suitable reagents for measuring metabolic activity also include PRESTOBLUE™ and ALAMARBLUE®, which can be used for fluorescence- or absorption-based microplate assays that measure the reductive capacity of cells. Metabolic activity can also be assessed by measuring glycolysis on the basis, for example, of the extracellular acidification rate. In some cases, protein content can be measured (e.g., via Bicinchoninic acid (BCA) assay, modified Lowry assay, ultraviolet absorbance measurement (e.g., 280 nm), Coomassie assay (Bradford assay), and the like) as a means of measuring metabolic activity, because protein content is proportional to metabolic activity. As such, an increase of protein content indicates an increase in metabolic activity. Additional suitable methods to measure metabolic activity include measuring: conversion of non-fluorescent Calcein AM to a fluorescence-emitter after acetoxymethyl ester hydrolysis by intracellular esterases; function of ion pumps and channels detected by fluorescence; change in pH indicators detected by fluorescence; and cellular and mitochondrial metabolism detected by measuring ATP using luminescence methodology. For more information about measuring metabolic activity, please see, for example, Zhang et al, Nat Protoc. 2012 May 10;7(6):1068-85: “Measuring energy metabolism in cultured cells, including human pluripotent stem cells and differentiated cells”, which is hereby incorporated by reference in its entirety.

### Viability

In some cases, a cell culture composition of the present disclosure provides for an at least 10%, at least 20%, at least 25%, at least 50%, at least 75%, at least 2-fold, at least 5-fold, or at least 10-fold, or more than 10-fold, increased viability of a cell. Such a change can be measured, for example, relative to a control cell (e.g., a cell cultured in the absence of a subject cell culture composition (e.g., cultured in the absence of ENPs)).

Methods to measure viability of a cell (cell viability) are known to one of ordinary skill in the art and any convenient method can be used. Two exemplary non-limiting methods to determine viability are metabolic viability assays, and dye exclusion viability assays.

Metabolic viability assays do not rely on the assumption that the cell membrane must lose its integrity in order to determine whether a cell is alive or dead. Metabolic viability assays usually rely on the cell's ability to perform a specific biochemical reaction that can be measured usually by absorbance, fluorescence or luminescence methods. The assays described above for measuring metabolic activity can also be interpreted as assays for measuring cell viability. As such, suitable assays for measuring cell viability include measuring: reduction of a tetrazolium compound, e.g. MTT/XTT and measured by absorbance; conversion of non-fluorescent Calcein AM to a fluorescence-emitter after acetoxymethyl ester hydrolysis by intracellular esterases; function of ion pumps and channels detected by fluorescence; change in pH indicators detected by fluorescence; and cellular and mitochondrial metabolism detected by measuring ATP using luminescence methodology.

Dye exclusion viability assays use a dye or stain that can enter the cell and usually intercalates with the DNA in the nucleus. The mere entry of the dye into the cell assumes that the cell membrane has lost its integrity and that the cell is dead. In other words, live cells exclude the dye, while dead cells allow the dye to enter. Dye or stains that are used in dye exclusion viability assays include, but are not limited to: (i) Trypan blue, which is often used to detect both viability and cell number using a hemocytometer or similar in an automated instrument; (ii) Propidium iodide (PI), which can be detected using manual and automated techniques, including a flow cytometer (PI is also used for cell cycle analysis and other assays); (iii) 7-Aminoactinomycin D (7-AAD), which can be detected by flow cytometry; and (iv) Acridine Orange, which can be used in hemocytometer procedures, but can also be detected by flow cytometry.

### Cell Proliferation

In some embodiments, a cell culture composition of the present disclosure does not decrease the proliferation of cells cultured in vitro in such cell culture composition, compared to the proliferation of the cells grown in the culture composition in the absence of the ENPs. For example, in some cases, a cell culture composition of the present disclosure decreases cell proliferation less than 10% (e.g., less than 5%, less than 3%, less than 2%, less than 1%, less than 0.5%, or not at all (0%)) relative to the proliferation of the cells grown in the culture composition in the absence of the ENPs. Such a change can be measured, for example, relative to a control cell (e.g., a cell cultured in the absence of a subject cell culture composition (e.g., cultured in the absence of ENPs)).

Methods to measure cell proliferation will be known to one of ordinary skill in the art and any convenient method can be used. However, care must be taken when selecting a method to measure cell proliferation. It is common in the art to use a cell viability assay/metabolic activity assay (as described above) as a proxy for cell proliferation because only metabolically active cells proliferate. However, as demonstrated in the examples section of this disclosure, metabolic activity can increase in cases where true cell proliferation stays the same and/or decreases. Thus, using an assay of metabolic activity as a proxy for cell proliferation is not suitable in methods that use the compositions of this disclosure. Suitable proliferation assays can measure cell features more directly associated with cell proliferation, such as DNA content, DNA synthesis, and cell number (directly counting cell number/cell concentration).

Because cellular DNA content is highly regulated, the DNA content assays can be used at multiple time points to calculate the average proliferation rate of a cell population. Any DNA dye that measure DNA content, e.g., PI, can be used. One non-limiting example of a suitable cell proliferation assay based on DNA content is the CyQUANT® cell proliferation assay provides a fluorescence microplate-based method for accurately counting cells in a population.

In one exemplary method, a DNA histogram (generated from DNA content assays) is used to observe effects on cell proliferation. By revealing the fraction of cells in the G1, S, and G2/M phases of the cell cycle (different regions of the histogram represent different phases), a DNA histogram can provide information about the relative fraction of resting and proliferating cells in a cell population.

DNA synthesis assays can employ DNA analogs (e.g., thymidine analogs such as bromo-deoxyuridine(BrdU), chloro-deoxyuridine(CldU), iodo-deoxyuridine (IdU), EDU (5-ethynyl-2′-deoxyuridine), and the like), which are incorporated only into those cells actively undergoing DNA synthesis, and therefore proliferating, at the time that the analog is available. Detecting of analog incorporation can include analog-specific antibodies (e.g., in the case of BrdU, and IdU); or “click” chemistry (e.g., in the case of EDU), which relies on a copper catalyzed covalent reaction between an azide and an alkyne.

### EUDRAGIT® Nanoparticles (ENPs)

EUDRAGIT® is the brand name for a diverse range of polymethacrylate-based copolymers. It includes anionic, cationic, and neutral copolymers based on methacrylic acid and methacrylic/acrylic esters or their derivatives.

In some embodiments, the EUDRAGIT® polymer included in an ENP in a subject cell culture composition is a EUDRAGIT® RS type polymer. A EUDRAGIT® RS type polymer comprises at least 60% by weight, e.g., 85% to 95% by weight, one or more (meth)acrylate copolymers from free-radical polymerized monomer units consisting of 93% to 98% by weight C₁- to C₄-alkyl esters of acrylic acid or of methacrylic acid and 7% to 2% by weight (meth)acrylate monomers having a quaternary ammonium group in the alkyl radical. The CAS number of EUDRAGIT® RS is 33434-24-1. The Chemical/IUPAC name of EUDRAGIT® RS is Poly(ethyl acrylate-co-methyl methacrylate-co-trimethylammonioethyl methacrylate chloride) 1:2:0.1. The ammonium groups are present as salts and make the polymers permeable. EUDRAGIT® RS 100 is granular, EUDRAGIT® RS PO is powder, EUDRAGIT® RS 30 D is a 30% aqueous dispersion, and EUDRAGIT® RS 12.5 is a 12.5% organic solution.

Methods of making EUDRAGIT® RS type polymer nanoparticles (ENPs) are known to one of ordinary skill in the art and any convenient method can be used. Two exemplary, but non-limiting, methods are nanoprecipitation (NP) and double emulsion (DE). For examples, see Fessi et al, 1989, Int. J. Pharm. 55, 1-4; ;and Bodmeier et al, 1991, J.Microencapsul.8,161-170, which are hereby incorporated by reference for their teachings on nanoparticle preparation. The nanoprecipitation technique usually produces smaller nanoparticles than the double emulsion technique, and is more likely to produce nanoparticles of the appropriate size for the subject compositions and methods (see more details regarding suitable nanoparticles sizes below).

### Nanoprecipitation

When performing the nanoprecipitation technique, polymer (e.g., EUDRAGIT® RS) is dissolved in organic phase (e.g., acetone). The organic solution is then flowed slowly (e.g. by pouring in the body of a syringe), while stifling, into a solution of block copolymer (e.g., Pluronic® F68 (0.5%,w/v)) in aqueous phase. The solvent is removed (e.g., by rotary evaporation under vacuum) until a suspension of nanoparticles is obtained. The resulting nanoparticles are nanoparticles of the polymer used in the first step of the process above (e.g., nanoparticles of a EUDRAGIT® RS polymer, i.e., ENPs).

### Double Emulsion

When performing the double emulsion technique (also known as solvent evaporation technique (w/o/w); and 2-step emulsification), polymer (e.g., EUDRAGIT® RS) is dissolved in organic phase (e.g., ethylacetate, methylene chloride, and the like). An aqueous solution (e.g., water) is emulsified into the organic phase by homogenization (e.g., via sonication , e.g., using an ultrasonic homogenizer). This primary water-in-oil (w/o) emulsion is then dispersed (homogenized , e.g., by sonication) into an aqueous solution of PVA (polyvinyl alcohol))(e.g., PVA at 0.1%, w/v (weight per volume)), thus producing a secondary water-in-oil-in-water emulsion (w/o/w) emulsion. The resulting nanoparticles can be obtained by removing the organic phase (e.g., via evaporation of the organic phase). The nanoparticles can be isolated (e.g., via centrifugation), washed (e.g., with deionized water), and stored (e.g., via freeze-drying). The resulting nanoparticles are nanoparticles of the polymer used in the first step of the process above (e.g., nanoparticles of a EUDRAGIT® RS polymer, i.e., ENPs).

Evaluating nanoparticles

After making nanoparticles, measurements can be performed to determine average particle size and/or zeta potential.

Methods of measuring nanoparticle size will be known to one of ordinary skill in the art and any convenient method can be used. One non-limiting example is via photon correlation spectroscopy (PCS) (also known as dynamic light scattering) (e.g., using a ZETASIZER™), which can be used to determine the size distribution profile of small particles in suspension or polymers in solution. Average particle size may also be determined using transmission electron microscopy.

The average diameter of the EUDRAGIT® RS (e.g., EUDRAGIT® RS 100) nanoparticles in a suitable population of EUDRAGIT® RS nanoparticles for use in a subject cell culture composition is in the range of from 30 nanometers (nm) to 100 nm, e.g., from 30 nm to 80 nm, from 30 nm to 70 nm, from 40 nm to 80 nm, from 50 nm to 80 nm, from 50 nm to 90 nm, from 50 nm to 100 nm, from 55 nm to 75 nm, from 55 nm to 80 nm, from 55 nm to 90 nm, from 55 nm to 100 nm, from 60 nm to 70 nm, from 62 nm to 68 nm, from 64 nm to 66 nm, from 60 nm to 80 nm, from 60 nm to 90 nm, or from 60 nm to 100 nm.

The zeta potential is the electrostatic potential at the electrical double layer surrounding a nanoparticle in solution. Zeta potential is one of the main forces that mediate interparticle interactions. Nanoparticles with a zeta potential between −10 and +10 mV are considered approximately neutral, while nanoparticles with zeta potentials of greater than +30 mV or less than −30 mV are considered strongly cationic and strongly anionic, respectively. Since most cellular membranes are negatively charged, zeta potential can affect a nanoparticle's tendency to permeate membranes.

Methods of measuring zeta potential will be known to one of ordinary skill in the art and any convenient method can be used. For example, particle electrophoretic mobility can be determined by laser Doppler electrophoresis (LDE). When using LDE, zeta potential is measured by applying an electric field across the dispersion. Particles within the dispersion with a zeta potential will migrate toward the electrode of opposite charge with a velocity proportional to the magnitude of the zeta potential. This velocity is measured using the technique of laser Doppler anemometry. The frequency shift or phase shift of an incident laser beam caused by these moving particles is measured as the particle mobility, and this mobility is converted to the zeta potential. Zeta potential can be measured, for example, using a microelectrophoresis cell of ZETASIZER™. For information about measuring zeta potential, see Clogston et al, Methods Mol Biol. 2011;697:63-70 , which is hereby incorporated by reference in its entirety.

EUDRAGIT® nanoparticles suitable for use in a subject cell culture composition have an average zeta potential in the range of from +30 millivolts (mv) to +80 mv, e.g., from +35 mv to +75 mv, from +40 mv to +70 mv, from +45 mv to +65 mv, from +45 mv to +60 mv, or from +47 mv to +55 mv. Standard cell culture components

Standard cell culture components that are suitable for inclusion in a subject cell culture composition include, but are not limited, to, a vitamin; an amino acid (e.g., an essential amino acid); a pH buffering agent; a salt; an antimicrobial agent (e.g., an antibacterial agent, and antimycotic agent, etc.); serum; an energy source (e.g., a sugar); a nucleoside; a lipid; trace metals; a cytokine, a growth factor, a stimulatory factor, and the like. Any convenient cell culture media can be used, and as is known in the art, various cell types grow better in particular media preparations (in some cases, particular media formulations have been optimized to culture specific types of cells (e.g., neurons, cardiomyocytes, hepatocytes, etc.). Accordingly, any convenient cell culture media can be used and may be tailored to the particular cell type being cultured.

In some embodiments, a subject cell culture composition includes animal serum (e.g., fetal bovine serum (FBS); bovine serum, chicken serum, newborn calf serum, rabbit serum, goat serum, normal goat serum (NGS); horse serum; lamb serum, porcine serum, and the like). A wide range of serum concentrations can be used. A cell culture composition of the present disclosure can have a concentration of serum in a range of from 1% to 50% (e.g., from 2% to 40%, from 2% to 30%, from 2% to 25%, from 2% to 20%, from 2% to 15%, from 2% to 10%, from 2% to 7%, from 2% to 5%, from 3% to 12%, from 5% to 15%, from 8% to 12%, from 8% to 20%, about 5%, about 6%, about 7%, about 8%, about 9%, about 10%, about 11%, about 12%, about 13%, about 14%, or about 15%). In some embodiments, a subject cell culture composition is serum free. Serum comprises growth factors and in many cases, is it unknown exactly which growth, or exactly how many growth factors are present in any given serum. In some cases, at least one of the growth factors present in a serum is known.

In some cases, a subject cell culture composition comprises a conditioned medium (e.g., in some cases, a subject cell culture composition comprises compounds (e.g., proteins, chemicals, etc.) that were secreted into the medium by cultured cells). Without being bound by theory, it is believed that when subject ENPs come in contact with conditioned medium, they increase the effective local concentration at the cell surface of compounds that are present in the medium (e.g., compounds that were secreted into the medium by cells). Thus, a medium can be conditioned by any cell type desired (e.g., a cell engineered to overexpress a secreted growth factor) and when combined with subject ENPs, the ENPs can cause an increase in local concentration of factors within the medium (e.g., a secreted growth factor). Cells that then come in contact with a conditioned medium comprising ENPs (or ENPs that were pre-incubated in conditioned medium) will be exposed to high local concentrations of factors that were present in the conditioned medium (e.g., the secreted growth factors). In some embodiments, ENPs are contacted with conditioned medium that includes known factors (e.g., a secreted factor from a genetically modified cell, a known factor secreted by a known cell type, etc.). In some embodiments, ENPs are contacted with conditioned medium that includes unknown factors.

A cell culture composition of the present disclosure can further include one or more of a cytokine; a growth factor; and/or any other convenient cell stimulatory factor. Such factors will be known to one of ordinary skill in the art, and any convenient factor can be used. In some cases, a cytokine, growth factor, and/or other cell stimulatory factor is provided as part of the serum (in cases of serum-containing media where the serum comprises a cytokine, growth factor, or other stimulatory factor). In some cases, a cytokine, growth factor, and/or other cell stimulatory factor can be provided by direct supplementation (e.g., can be added directly to the medium). In some cases, a cytokine, growth factor, or other stimulatory factor can be provided by contacting the medium with a cell or a population of cells that secretes the cytokine, growth factor, or other stimulatory factor into the medium. In some such cases, the medium can be considered conditioned medium (discussed above).

A cell culture composition of the present disclosure can have a pH in a range of from 6.8 to 7.4, e.g., 6.8 to 7.0, 7.0 to 7.2, or 7.2 to 7.4.

The major ions and their concentrations in cell culture media are generally present in standard, commercially available, liquid culture media (e.g., basal liquid culture media). Most standard types of media (e.g., DMEM, DMEM/F12. BME, RPM 1640, and the like) use relatively narrow and fixed ranges for the concentrations of bulk ions in general and the monovalent cations Na⁺ and K⁺ in particular. This is in line with the fact that the ionic balance of the bulk ions in general and the monovalent cations Na⁺ and K⁺ in particular is a rather universal property of almost all mammalian cells. Any convenient media that can be used to culture cells in vitro is suitable for use with the subject compositions and methods.

In accordance with the typical concentration of sodium ions inside and outside a generic mammalian cell (Alberts et al., Molecular Biology of the Cell (1994)) mostly sodium concentrations of about 145 mM are chosen together with potassium ion concentrations of around 5 mM. For most media types this results in a ratio between sodium and potassium ions that ranges between about 20-30 (see U.S. Pat. No. 5,135,866; and US Patent Publication No. 2013/0122543, both of which are hereby incorporated by reference in their entirety).

The osmolality of the media at the beginning of culturing is typically between about 280 and about 365 mOsm, but may also gradually increase during culturing and the addition of feeding solutions to values of less than or about 600 mOsm/kg. A cell culture composition of the present disclosure can have an initial osmolality in a range of from 200 to 400 mOsm/kg (e.g., from 200 to 400 mOsm/kg, from 250 to 375 mOsm/kg, or from 275 to 350 mOsm/kg).

### Methods of Culturing Cells In Vitro

The present disclosure provides a method for culturing a cell in vitro, the method comprising culturing the cell in a subject cell culture composition. In some cases, a method of culturing is referred to as a method of increasing cellular metabolism (e.g., in a cell), a method of increasing the metabolic activity of a cell, a method of increasing cellular growth, and/or a method of increasing growth of a cell.

The methods generally involve culturing a cell in a liquid cell culture composition comprising a population of at least two EUDRAGIT® RS nanoparticles (ENPs). Without being bound by theory, the ENPs may increase the effective local concentration at the cell surface of growth factors and/or other factors present in cell culture medium. In some cases, the average diameter of the nanoparticles of the population is from 30 nm to 100 nm, e.g., from 30 nm to 80 nm, from 30 nm to 70 nm, from 40 nm to 80 nm, from 50 nm to 80 nm, from 50 nm to 90 nm, from 50 nm to 100 nm, from 55 nm to 75 nm, from 55 nm to 80 nm, from 55 nm to 90 nm, from 55 nm to 100 nm, from 60 nm to 70 nm, from 62 nm to 68 nm, from 64 nm to 66 nm, from 60 nm to 80 nm, from 60 nm to 90 nm, or from 60 nm to 100 nm.

A subject method generally involves culturing a cell in vitro in a subject liquid culture medium comprising EUDRAGIT® RS nanoparticles. In some embodiments, a subject method involves contacting a cell with a liquid culture medium in vitro, so that the culture medium comprises the cell; and contacting the culture medium comprising the cell with a population of ENPs. In some cases, the ENPs are naked (i.e., unloaded) nanoparticles. In some cases, a cell is contacted with the ENPs for a period of time of from 4 hours to 72 hours (e.g., from 12 hours to 72 hours, from 24 hours to 72 hours, from 4 hours to 12 hours, from 12 hours to 24 hours, from 24 hours to 48 hours, or from 48 hours to 72 hours). In some cases, a cell is cultured in a liquid culture medium comprising ENPs for a period of time of more than about 12 hours, more than about 24 hours, more than about 36 hours, or more than about 48 hours. Cells cultured using the subject methods generally exhibit an increased metabolic activity. In some cases, the methods further include measuring the metabolic activity of contacted cells.

In some cases, the ENPs contact a cell culture medium in the absence of cells. Thus, in some cases, the ENPs are added to the media prior to contacting a subject cell (i.e., the nanoparticles are added to the media before cell seeding). In some cases, the nanoparticles contact a culture medium after cells have already been in contact with the culture medium. Thus, in some such cases, the ENPs are added to the media after cell seeding. In some cases, the ENPs contact a subject cell at the same time that the cell contacts fresh culture medium. Thus, in some such cases, the ENPs are added to the media simultaneous with cell seeding. Therefore, subject nanoparticles can contact a cell culture medium before, after, or simultaneous with cell seeding.

### Cells

Cells that can be cultured using a cell culture composition of the present disclosure include primary cells and immortalized cell lines. In some cases, the cells are not immortalized cells. In some cases, the cell is a cancer cell.

Suitable cells can include, but are not limited to, primary and/or immortalized: cells of the nervous system (e.g., neurons, neural progenitor cells, astrocytes, oligodendrocytes, and the like); epithelial cells; endothelial cells; fibroblasts; chondrocytes; osteoblasts; osteoclasts; hepatocytes; pericytes; muscle cells; cardiomyocytes; smooth muscle cells; skeletal muscle cells; myoepithelial cells; satellite cells; stem cells (e.g., pluripotent stem cells, embryonic stem cells, induced pluripotent stem cells, germ cells, hematopoietic stem cells, etc.); progenitor cells (e.g., neural progenitor cells); fat cells, T cells; B cells, Mast cells, reticuloctyes, neutrophils, macrophages, dendritic cells, microglia, and B lymphocytes. Suitable cells can also include cancer cells of any of the above cell types (e.g., primary cancer cells, breast cancer cells, epithelial cancer cells, hematopoietic stem cell cancer cells etc.).

In some embodiments, the subject cells are mammalian cells. In some cases, suitable mammalian cells can include cells of the primary human breast cell line (HMEC 184), human breast cancer lines (e.g., MCF-7, MDA- MB-231, and the like), BALB/c mouse myeloma line, human retinoblasts (PER.C6), monkey kidney cells, human embryonic kidney line (HEK293), baby hamster kidney cells (BHK), Chinese hamster ovary cells (CHO) (e.g., CHO, CHO-K1, CHO-DG44, or CHO-DUX cells), mouse sertoli cells, African green monkey kidney cells (e.g., VERO-76, COS-7), human cervical carcinoma cells (HeLa), canine kidney cells, buffalo rat liver cells, human lung cells, human liver cells, mouse mammary tumor cells, TR1 cells, MRC 5 cells, FS4 cells, and/or or human hepatoma line (Hep G2).

In some embodiments, a cell cultured using a subject method is a non-phagocytic cell (i.e., the cell is not a phagocytic cell). Phagocytic cells generally include professional phagocytes such as, for example, neutrophils, mast cells, macrophages, and dendritic cells. Macrophages can include microglia, alveolar macrophages, osteoclasts, histiocytes, Kupffer cells, intestinal macrophages, adipose tissue macrophages, and the like. In some cases, a cell cultured using a subject method is not a macrophage. In some cases, a cell cultured using a subject method is not a macrophage or a neutrophil. In some cases, a cell cultured using a subject method is not a macrophage or a dendritic cell. In some cases, a cell cultured using a subject method is not a macrophage, a neutrophil, or a dendritic cell. In some cases, a cell cultured using a subject method is not a macrophage, a neutrophil, a dendritic cell, or a microglial cell.

Accordingly, in some embodiments, suitable cells can include, but are not limited to, primary and/or immortalized: cells of the nervous system (e.g., neurons, neural progenitor cells, astrocytes, oligodendrocytes, and the like); epithelial cells; endothelial cells; fibroblasts; chondrocytes; osteoblasts; hepatocytes; pericytes; muscle cells; cardiomyocytes; smooth muscle cells; skeletal muscle cells; myoepithelial cells; satellite cells; stem cells (e.g., pluripotent stem cells, embryonic stem cells, induced pluripotent stem cells, germ cells, hematopoietic stem cells, etc.); progenitor cells (e.g., neural progenitor cells); fat cells, T cells; B cells, and reticuloctyes. Suitable cells can also include cancer cells of any of the above cell types (e.g., primary cancer cells, breast cancer cells, epithelial cancer cells, hematopoietic stem cell cancer cells etc.).

In some embodiments, cells that are cultured using a subject method are primary cells. In some embodiments, cells that are cultured using a subject method are epithelial cells. Epithelial cells include, but are not limited to, pancreatic epithelial ductal cells (e.g., isolated from pancreatic tissue); prostate epithelial cells (isolated from prostate tissue); kidney epithelial cells (e.g., isolated from kidney tissue); breast epithelial cells (e.g., a breast cancer cell); and the like. In some embodiments, cells that are cultured using a subject method are primary epithelial cells. In some embodiments, cells that are cultured using a subject method are cancer cells (e.g., epithelial cancer cells, breast epithelial cancer cells, etc.).

A cell cultured using a subject method can be a cell from any organism. For example, a subject cell can be a cell from a plant; algae; an invertebrate (e.g., a cnidarian, an echinoderm, a worm, a fly, etc.); a vertebrate (e.g., a fish (e.g., zebrafish, puffer fish, gold fish, etc.), an amphibian (e.g., salamander, frog, etc.), a bird (e.g., chicken, turkey, etc.), a reptile (e.g., snake, lizard, etc.), a mammal, etc.). In some embodiments, the cells are mammalian cells, e.g., human cells; non-human primate cells; rodent (e.g., mouse; rat) cells; bovine cells; porcine cells; ungulate cells; feline cells; canine cells; or lagomorph (e.g., rabbit) cells.

### Culturing a Cell

Methods for culturing a cell in vitro (e.g., methods for increasing cellular metabolism, methods for increasing the metabolic activity of a cell, methods for increasing cellular growth, methods for increasing growth of a cell, and the like) are provided. In some embodiments, the subject method comprises culturing a cell in a culture composition comprising: a population of at least two EUDRAGIT® RS nanoparticles (ENPs) (i.e., nanoparticles of poly(ethyl acrylate-co-methyl methacrylate-co-trimethylammonioethyl methacrylate chloride) 1:2:0.1). In some such embodiments, the average diameter of the nanoparticles of the population is from 30 nm to 100 nm, e.g., from 30 nm to 80 nm, from 30 nm to 70 nm, from 40 nm to 80 nm, from 50 nm to 80 nm, from 50 nm to 90 nm, from 50 nm to 100 nm, from 55 nm to 75 nm, from 55 nm to 80 nm, from 55 nm to 90 nm, from 55 nm to 100 nm, from 60 nm to 70 nm, from 62 nm to 68 nm, from 64 nm to 66 nm, from 60 nm to 80 nm, from 60 nm to 90 nm, or from 60 nm to 100 nm. In some embodiments, the culture composition further comprises a growth factor. In some embodiments, the culture composition further comprises animal serum (e.g., fetal bovine serum, goat serum, and the like). In some embodiments, the culture composition comprises conditioned media.

In some embodiments, the subject method comprises (a) contacting a cell with a culture medium so that the culture medium comprises the cell; and (b) contacting the culture medium comprising the cell with a population of at least two EUDRAGIT® RS nanoparticles (ENPs) (i.e., nanoparticles of poly(ethyl acrylate-co-methyl methacrylate-co-trimethylammonioethyl methacrylate chloride) 1:2:0.1).

In some embodiments, the culture medium comprises a growth factor. In some embodiments, the culture medium comprises animal serum (e.g., fetal bovine serum, goat serum, and the like). In some embodiments, the culture medium is conditioned media. In some embodiments, the cell is contacted with a serum-free culture medium prior to contacting the culture medium with subject nanoparticles. In some embodiments, the cell is not contacted with a serum-free culture medium prior to contacting the culture medium with subject nanoparticles.

In some embodiments, the cell is contacted with a naked nanoparticle (e.g., a nanoparticle that is not previously loaded, e.g., not previously contacted with a compound that can be “loaded” onto to the nanoparticle). In some such cases the nanoparticles are naked prior to contacting the culture medium comprising the cell.

In some cases, the nanoparticles are loaded nanoparticles (e.g., the nanoparticles are/were loaded prior to contacting the culture medium comprising the cell). In some such cases, the nanoparticles are/were loaded by contacting a conditioned medium, a medium containing a growth factor, and/or a medium containing serum.

In some cases, the cell is contacted (e.g., via contacting the culture medium comprising the cell) with a nanoparticle for a period of time greater than 24 hours (e.g., greater than 36 hours, greater than 48 hours, greater than 60 hours, greater than 72 hours). Thus, the culture medium comprising the cell is contacted with a subject nanoparticle for a period of time greater than 24 hours (e.g., greater than 36 hours, greater than 48 hours, greater than 60 hours, greater than 72 hours, or greater than 96 hours).

In some cases, the cell is contacted with a nanoparticle for a period of time ranging from 24 hours (hrs) to 96 hrs (e.g., from 24 hrs to 84 hrs, from 24 hrs to 72 hrs, from 24 hrs to 60 hrs, from 24 hrs to 48 hrs, from 24 hrs to 36 hrs, from 36 hrs to 96 hrs, from 36 hrs to 84 hrs, from 36 hrs to 72 hrs, from 36 hrs to 60 hrs, from 36 hrs to 48 hrs, from 48 hrs to 96 hrs, from 48 hrs to 84 hrs, from 48 hrs to 72 hrs, or from 48 hrs to 60 hrs).

Thus, the culture medium comprising the cell is contacted with a subject nanoparticle for a period of time ranging from 24 hours (hrs) to 96 hrs (e.g., from 24 hrs to 84 hrs, from 24 hrs to 72 hrs, from 24 hrs to 60 hrs, from 24 hrs to 48 hrs, from 24 hrs to 36 hrs, from 36 hrs to 96 hrs, from 36 hrs to 84 hrs, from 36 hrs to 72 hrs, from 36 hrs to 60 hrs, from 36 hrs to 48 hrs, from 48 hrs to 96 hrs, from 48 hrs to 84 hrs, from 48 hrs to 72 hrs, or from 48 hrs to 60 hrs).

In some embodiments, the subject methods result in an increase in the metabolic activity of the cell that is contacted with the subject nanoparticles. In some cases, the method further comprises measuring the metabolic activity of the cell. In some cases, measuring the metabolic activity of the cell is performed using a tetrazolium dye other than MTT (3-(4,5-dimethylthiazol-2-yl)-2,5-diphenyltetrazolium bromide, a yellow tetrazole). In some embodiments, measuring the metabolic activity of the cell is performed using a tetrazolium dye selected from the group consisting of: XTT, MTS, WST-1, and WST-8.

Methods of Extracting Proteins from Serum

The present disclosure provides a method for extracting proteins from serum. The method generally involves contacting a serum sample with a population of subject ENPs for a period of time sufficient for the nanoparticles to bind to proteins present in the serum. The protein-bound nanoparticles are then isolated. In some embodiments, the extracted proteins provide for metabolic activity and/or growth of cells in culture; e.g., in some cases, the extracted proteins provide for enhanced metabolic activity and/or growth of cells in culture

Subject nanoparticles are EUDRAGIT® RS nanoparticles (ENPs). In some cases, the average diameter of the nanoparticles of the population is from 30 nm to 100 nm, e.g., from 30 nm to 80 nm, from 30 nm to 70 nm, from 40 nm to 80 nm, from 50 nm to 80 nm, from 50 nm to 90 nm, from 50 nm to 100 nm, from 55 nm to 75 nm, from 55 nm to 80 nm, from 55 nm to 90 nm, from 55 nm to 100 nm, from 60 nm to 70 nm, from 62 nm to 68 nm, from 64 nm to 66 nm, from 60 nm to 80 nm, from 60 nm to 90 nm, or from 60 nm to 100 nm.

The term “serum sample” as used herein refers to serum obtained by any convenient method known by one of ordinary skill in the art. A serum sample can be serum that is purchased from a commercial supplier, serum derived from a human (e.g., patient serum, adult serum, fetal serum, newborn serum, etc.), or serum derived from any animal. In some cases, a serum sample is a sample of serum selected from: human serum, fetal bovine serum (FBS); bovine serum, chicken serum, newborn calf serum, rabbit serum, goat serum; horse serum; lamb serum, porcine serum, and a combination thereof. Also of interest are biological fluids, which may contain serum proteins (e.g., cerebrospinal fluid (CSF), urine (an ultrafiltrate of serum), a transudate (e.g., a pathological transudate, a potentially pathological transudate), an exudate (e.g., a pathological exudate, a potentially pathological exudate), and the like). Accordingly, the term “serum sample,” as used herein, encompasses biological fluids that may contain serum proteins.

By “isolated”, “isolating”, etc. it is meant that the protein-bound nanoparticles are removed from contact with the serum sample. For example, the mixture of nanoparticles and serum can be centrifuged (e.g., at 2,500×g for 10 min) to pellet the nanoparticles, and the supernatant (protein-extracted serum sample) can be removed. In some cases, the nanoparticles are rinsed, stored, and/or analyzed in a suitable buffer (e.g, phosphate buffered saline (PBS), glycine•HCl, Tris•HCl/NaCl, guanidine thiocyanate (6M), etc.). Appropriate buffers for various applications will be known to one of ordinary skill in the art.

The subject methods include contacting a serum sample with a population of subject nanoparticles for a period of time sufficient for the nanoparticles to bind to proteins present in the serum or in a biological fluid. A suitable period of time is from 10 minutes (min) to 24 hours (hrs) (e.g., from 10 min to 60 min, from 10 min to 45 min, from 10 min to 30 min, from 20 min to 60 min, from 20 min to 45 min, from 20 min to 30 min, from 30 min to 8 hrs, from 30 min to 6 hrs, from 30 min to 4 hrs, from 30 min to 2 hrs, from 30 min to 60 min, from 1 hr to 24 hrs, from 1 hr to 12 hrs, from 1 hr to 6 hrs, from 1 hr to 4 hrs, or from 1 hr to 2 hrs).

In some cases, the extracted proteins are identified. Any convenient method may be used to identify the proteins bound to the subject nanoparticles, and various methods will be known to one of ordinary skill in the art.

As one non-limiting example, protein-bound nanoparticles can be contacted with guanidine thiocyanate (e.g., 6M), the proteins separated by size using SDS (sodium dodecyl sulfate) gel electrophoresis, and proteins can be prepared from the gel for mass spectrometry (e.g., MALDI-TOF mass spectrometry).

In some cases, an extracted protein comprises an amino acid sequence of an Interpro domain. Thus, in some cases, a subject method comprises identifying an Interpro domain of a protein bound to an isolated protein-bound nanoparticle. Interpro domains will be known to one of ordinary skill in the art and information regarding Interpro domains can be found at, for example, “www.ebi” followed by “.ac.uk/interpro/about.html”.

Exemplary Interpro domains that can be identified include, but are not limited to: IPR006210:EGF-like, IPR000215:Protease_inhib_I4_serpin, IPR013783:Ig-like_fold, IPR016060:Complement_control_module, IPR001254:Peptidase_S1_S6, IPR001611:Leu-rich_rpt, IPR006209:EGF, IPR008985:ConA-likelec_gl, IPR000436:Sushi_SCR_CCP, IPR009003:Pept_cys/ser_Trypsin-like, IPR018039: Intermediate_filament_CS, IPR011992:EF-hand-like_dom, PR002035:VWF_A, IPR001599:Macroglobln_a2, IPR000859:CUB, IPR008160:Collagen, IPR003961:Fibronectin_type3, IPR004001:Actin_CS, IPR000264:Serumumin, IPR000001:Kringle, IPR017857:Coagulation_fac_subgr_Gla_dom, IPR009053:Prefoldin, IPR018056:Kringle_CS, IPR000719:Prot_kinase_cat_dom, IPR001791:Laminin_G, IPR000010:Prot_inh_cystat, IPR000884:Thrombospondin_1_rpt, IPR012674:Calycin, IPR008979:Galactose-bd-like, IPR020837:Fibrinogen_CS, IPRO01304:C-typelectin, IPR002223:Prot_inh_Kunz-m, and IPR017441:Protein_kinase_ATP_BS.

In some cases, a subject method comprises identifying a protein bound to an isolated protein-bound nanoparticle. Examples of proteins that can be extracted using the subject methods include, but are not limited to: actin, gamma-enteric smooth muscle, kininogen-2 (isoform II), serpin A3-3, serpin A3-5, CD40 ligand , C-type lectin domain family 11 member A, talin-1, Ig heavy chain Mem5-like, partial, SERPINA3-8, protein S100-A10 , mitochondrial fission 1 protein, factor XIIa inhibitor, sphingomyelin phosphodiesterase, pleckstrin, keratin, type II cytoskeletal 7, collectin-43, serpin A3-7, ras-related protein Rab-6B, proheparin-binding EGF-like growth factor, tRNA guanosine-2′-O-methyltransferase TRM13 homolog, asporin, biglycan, zinc finger and BTB domain-containing protein 48, nucleolar GTP-binding protein, aspartyl-tRNA synthetase, cytoplasmic solute carrier family 2_facilitated glucose transporter member 4, leucine-rich repeat transmembrane neuronal protein 4, toll-like receptor 6, v-src sarcoma (Schmidt-Ruppin A-2) viral oncogene homolog, coiled-coil domain-containing protein 147, transmembrane and TPR repeat-containing protein 3, cylicin-1, GPI ethanolamine phosphate transferase 2, AFG3-like protein 2, dynamin-2, nebulette, structural maintenance of chromosomes proteinll, nardilysin, WD repeat-containing protein 17, tubulin polyglutamylase , TTLL5 isoform 1, citron Rho-interacting kinase, neurexin-2-beta, AT-rich interactive domain-containing protein 2, 5 carrying the IPR006210:EGF-like domain, LOC616876, LOC782051, LOC100139405, and C10orf18.

In some cases, the amount of a protein bound to a subject nanoparticle is measured. Any convenient method may be used (e.g., Enzyme-linked Immunosorbent Assay (ELISA), Western Blot, mass spectrometry, etc.). In some cases, the proteins are removed from protein-bound nanoparticles prior to identification.

In some embodiments, a subject method includes contacting a cell (e.g., contacting a culture medium that is in contact with a cell) with protein-bound nanoparticles (protein/ENP complexes), where the protein-bound nanoparticles were produced by a subject method of extracting proteins from a serum sample. The proteins bound to the protein/ENP complexes are in some cases non-covalently bound to the ENP. In some of these embodiments, the contacting is in a liquid culture medium or, for example, in a biological fluid (e.g., human body fluid). In some cases, the contacting is in a serum-free liquid culture medium. In some cases, the protein/ENP complexes comprise one or more of the following proteins: actin, gamma-enteric smooth muscle, kininogen-2 (isoform II), serpin A3-3, serpin A3-5, CD40 ligand , C-type lectin domain family 11 member A, talin-1, Ig heavy chain Mem5-like, partial, SERPINA3-8, protein S100-A10, mitochondrial fission 1 protein, factor XIIa inhibitor, sphingomyelin phosphodiesterase, pleckstrin, keratin, type II cytoskeletal 7, collectin-43, serpin A3-7, ras-related protein Rab-6B, proheparin-binding EGF-like growth factor, tRNA guanosine-2′-O-methyltransferase TRM13 homolog, asporin, biglycan, zinc finger and BTB domain-containing protein 48, nucleolar GTP-binding protein, aspartyl-tRNA synthetase, cytoplasmic solute carrier family 2_facilitated glucose transporter member 4, leucine-rich repeat transmembrane neuronal protein 4, toll-like receptor 6, v-src sarcoma (Schmidt-Ruppin A-2) viral oncogene homolog, coiled-coil domain-containing protein 147, transmembrane and TPR repeat-containing protein 3, cylicin-1, GPI ethanolamine phosphate transferase 2, AFG3-like protein 2, dynamin-2, nebulette, structural maintenance of chromosomes proteinll, nardilysin, WD repeat-containing protein 17, tubulin polyglutamylase , TTLL5 isoform 1, citron Rho-interacting kinase, neurexin-2-beta, AT-rich interactive domain-containing protein 2, 5 carrying the IPR006210:EGF-like domain, LOC616876, LOC782051, LOC100139405, and C10orf18.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Materials and Methods

### Preparation of EUDRAGIT™ RS Nanoparticles

The EUDRAGIT® RS 100 copolymer used in this study was purchased from Evonik Industries, and is a co-polymer of ethylacrylate and methylmethacrylate, with low methacrylic acid ester content with quaternary ammonium groups. EUDRAGIT® RS nanoparticles (ENPs) were prepared as described previously (Eidi et al., 2010) by dissolving a co-polymer of ethylacrylate and methylmethacrylate in acetone (20 mg/mL). The organic solution was poured in a syringe, flowed under stifling in 40 mL of a Pluronic® F68 (0.5%, w/v) aqueous phase. The solvent was removed by rotary evaporation under vacuum at 40° C. to a final polymer concentration of 7.5 mg/mL. The obtained nanoparticles displayed a monodispersed distribution at 65.0±26.3 nm and a Z-average of +51.04 mV. Their refractive index was 1.59, the viscosity was 0.8872 cP, and the relative density (d₂₀²⁰) ranged from 0.816 to 0.836. The concentration of Pluronic F68 was 0.0008% for 200 μg/ml concentration of polymer. The potential effect of this concentration of Pluronic F68 on cells was evaluated, and none was observed.

### Metabolic Activity Assay WST-1

Human breast cancer cell lines (MCF-7, MDA- MB-231) and the primary human breast cell line (HMEC 184) were grown in their respective media as described previously (Hussien & Brooks, 2011). Cells were seeded at 2,000 per well and grown at 37° C. in an air/CO₂ atmosphere (95/5 v/v) for 24, 48, and 72 h in the presence of 0, 3.1, 6.2, 12.5, 25, 50, 100, or 200 μg/mL ENPs in 200 μl of their respective media. At each time point, the medium was discarded, cells were washed with PBS 1×, and DMEM medium without phenol red (supplemented with 10% FBS (fetal bovine serum), 1% L-glutamine, and 0.25% penicillin-streptomycin) but containing WST-1 (Roche assay), was added and cells were incubated for 1 to 5 h. The 96-well plates were then read by spectrophotometry at 450 nm.

### BrdU and EdU Cell Proliferation Assays

Cells were seeded in 96-well plates at 2,000 per well and grown in a T.C. incubator at 37° C. and 5% CO₂ for 24 h in the presence of ENPs in 200 μl of their respective media. 5-bromo-2′-deoxyuridine (BrdU) was used to label proliferating cells with a BrdU cell proliferation assay (Calbiochem, Darmstadt, Germany). Cells were incubated with BrdU for 6 h, then fixed with fixing/denaturing solution. The BrdU-labeled DNA was detected with a BrdU Mouse mAb kit using the manufacturer's protocol. The absorbance in each plate was measured using a spectrophotometer at dual wavelengths of (450-540 nm). To quantify the percentage of proliferating cells and total cell count after 24 h, HMEC 184 cells were seeded (5,000 per chamber) in an eight-chamber slide (Lab-tek, Pa., USA) and treated with 0, 6, 25, and 100 μg/ml ENPs for 24 h. Cells were incubated with 5-ethynyl-2′-deoxyuridine (EdU) (30 μM) from Invitrogen for 6 h, and then washed twice with PBS and fixed with 4% paraformaldehyde. Cells were permeabilized with 0.1% Triton-X 100 in PBS for 5 min, then incubated with 5% NDS blocking buffer for 2 h. Incorporated EdU was detected with a copper-catalyzed fluorescent azide reaction (Click-iT, Invitrogen), after which slides were washed with PBS and mounted on cover slips with mounting medium containing DAPI (Vector, Burlingame, Calif., USA). Nuclei and EdU positive nuclei were counted using the (20×/0.8 NA) air objective of an Axio Observer.Z1 fluorescence microscope (Zeiss, Germany) with Metamorph software (Molecular Devices, Calif., USA).

### Confocal Laser Scanning Microscopy and Phase-Contrast Microscopy

ENPs were conjugated with Nile red (nominal diameter=73 nm, zeta potential =+47 and polydispersity index=0.34) according to a method described previously (Yoo et al., 2011). Nuclei were stained with Hoechst 33342 (Sigma), mitochondria were stained with MitoTracker Deep Red 633 (Invitrogen/Molecular Probes, Eugene, Oreg.), and the membranes were stained with Wheat Germ Agglutinin Alexa Fluor 488 conjugate, WGA (Invitrogen). Cells were observed with a (20×/1.0 NA) water-dipping objective in a Zeiss LSM 780 microscope. A 3D movie of HMEC 184 cells incubated for 3 days with Nile red-ENPs was generated using Imaris software (Bitplane, Zurich, Switzerland). ImageJ software was used to find ENPs localized with mitochondria (Wayne Rasband, NIH, Bethesda, Md., USA). The average mitochondrial volume of single HMEC 184 cells stained with Mito Tracker was measured using Imaris software from 3D images.

### Total Protein Measurement and Immunoblots

Cells were washed twice with PBS and then solubilized with 5% NP-40. Total protein concentration was determined using a BCA protein assay kit (Pierce Biotechnology, Radford, Ill.). Western blotting was performed as previously described (Hussien & Brooks, 2011). Primary antibodies used were rabbit anti-cytochrome oxidase subunit IV, mouse anti-β-actin, and mouse anti-VDAC (Abcam, Cambridge, Mass.). Band intensity was quantified as previously described (Hussien & Brooks, 2011).

Purification & Identification Of Serum Proteins Coated on Enps with Proteomic Mass Spectrometry

A mixture of serum and ENPs was centrifuged at 2,500 g for 10 min and the obtained pellet was washed twice with PBS and exposed to organic extraction with dichloromethane. The organic phase was examined on thin layer chromatography (TLC), and the aqueous phase was examined with UV/VIS spectrometry. To identify the proteins attached to ENPs, FBS (5.9 mL, approx. 21.44 mg of proteins) containing 780 μg/mL of ENPs was centrifuged at 10,000 g for 3.5 min. The pellet was washed twice with an equal volume of PBS ×1, then resuspended in 1 mL of either glycine•HCl (100 mM, pH=3), Tris•HCl/NaCl (50 mM/5 M, pH=8), or guanidine thiocyanate (6M). The three samples were run on SDS (sodium dodecyl sulfate) gel electrophoresis and stained with Coomassie Brilliant Blue. Seven bands from the SDS gel of guanidine thiocyanate were examined with MALDI-TOF mass spectrometry, which identified 290 non-redundant proteins belonging to Bos taurus, and nine human contaminant cytokeratins. A total of 178 proteins were identified and analyzed for name of product in Bos taurus (as they appeared in Unigene “www” followed by “.ncbi.nlm.nih.gov” followed by “/UniGene/”), name of gene in Homo sapiens counterpart, name of human counterpart protein (“www” followed by “.uniprot.org/” followed by “uniprot/”), and plasma levels in human and InterPRO domains if applicable (“www” followed by “.genecards.org/”). A total of 69 proteins were cited only by their Bos taurus name and were not included in data analysis because either (i) their relative abundance (RA) was very low (1 to 10), (ii) their identified peptides span less than 3% of the protein sequence, or (iii) they were isoforms of, or closely related to, already analyzed proteins. Proteins were ranked according to their (i) abundance (A), namely the ratio of spectrum count/length, and (ii) sequence coverage (SC), namely the percentage of the entire sequence that was expressed in the peptides found in trypsin hydrolysate. The relative abundance (RA) was calculated as the ratio of the most abundant protein to the least abundant protein. The InterPro domains (“www.” followed by “ebi.ac.” followed by “uk/interpro/”) of 178 proteins were retrieved and were submitted to the STRING database (“string-” followed by “db.org/”).

Total RNA Extraction and microarray analysis

Total RNA was extracted from HMEC 184 cells (50% and 90% confluence) incubated with 25 μg/mL ENPs for 24 h, and without incubation (control). The quality of RNA extracted with RiboPure kit (Ambion, Austin, Tex.) was determined with spectrophotometry and capillary electrophoresis, using RNA 6000 Nano® (Agilent 2100 Bioanalyser™). cDNA synthesis, cRNA synthesis, Cy3-dye labeling, and microarray hybridization were carried out using 100 ng of total RNA according to manufacturer protocol (One-Color Microarray-Based Gene Expression Analysis, version 6.6). Microarray slides (SurePrint G3 Human GE v2 8×60K, Agilent technologies) were scanned with an Agilent DNA microarray scanner. The acquisition, quantification of array images, and primary data analysis were performed using Agilent Feature Extraction Software. Data were first normalized with quantile method and stringent filtering criteria were next used to identify genes whose expression level was significantly changed, with a modified Student t-test (p 0.001) and FC (fold change) ≧2.0. FC of mean of three replicates (for each ENP exposure and cell condition) on control were calculated. The selected genes display acceptable False Discovery Rate (<15%). The Database for Annotation, Visualization, and Integrated Discovery (DAVID; “david.abcc” followed by “.ncifcrf.gov”) was then used to analyze and extract (i) relevant GO terms (“godatabase.” followed by “org”) (ii) functions and expression data on Genecard (“genecards.” followed by “org”) and (iii) known and predicted protein-protein interactions (“string-” followed by “db.org”) for selected genes. The raw data of our microarrays are available on “ncbi.nlm.nih.” followed by “gov/geo/”, using the GSE45598 and GSE45869 access number.

### Statistical Analysis

Testing for significant differences between groups at p<0.05 was done either by the student's t test, one-way ANOVA analysis with Tukey's post test (comparing all groups), or Dunnett's post test (comparing all groups vs. control) using RLPlot or Prism software.

### RESULTS

### Dose-Dependent Increases in Metabolic Activity of Epithelial Cells Exposed to ENPs

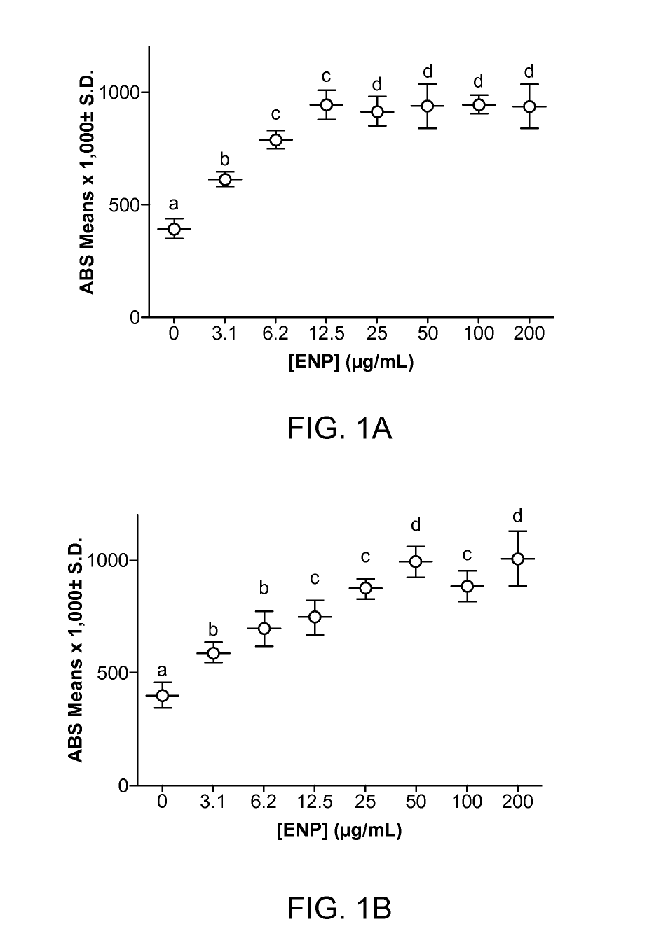

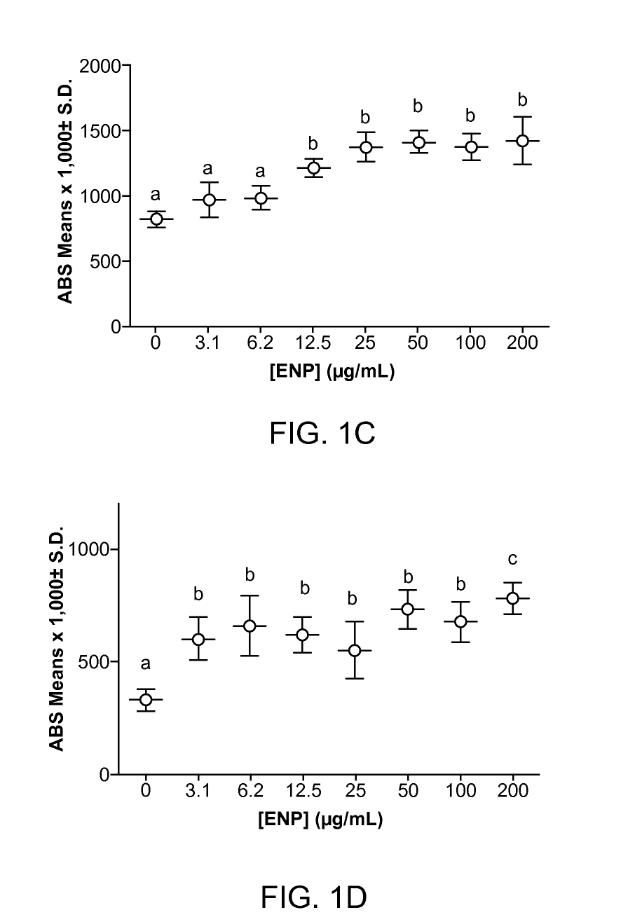

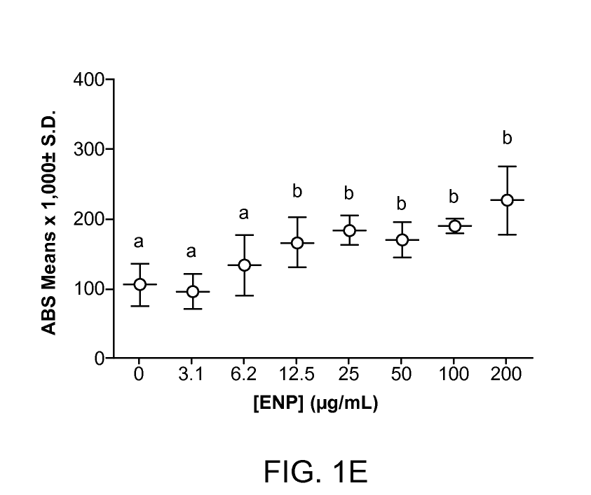

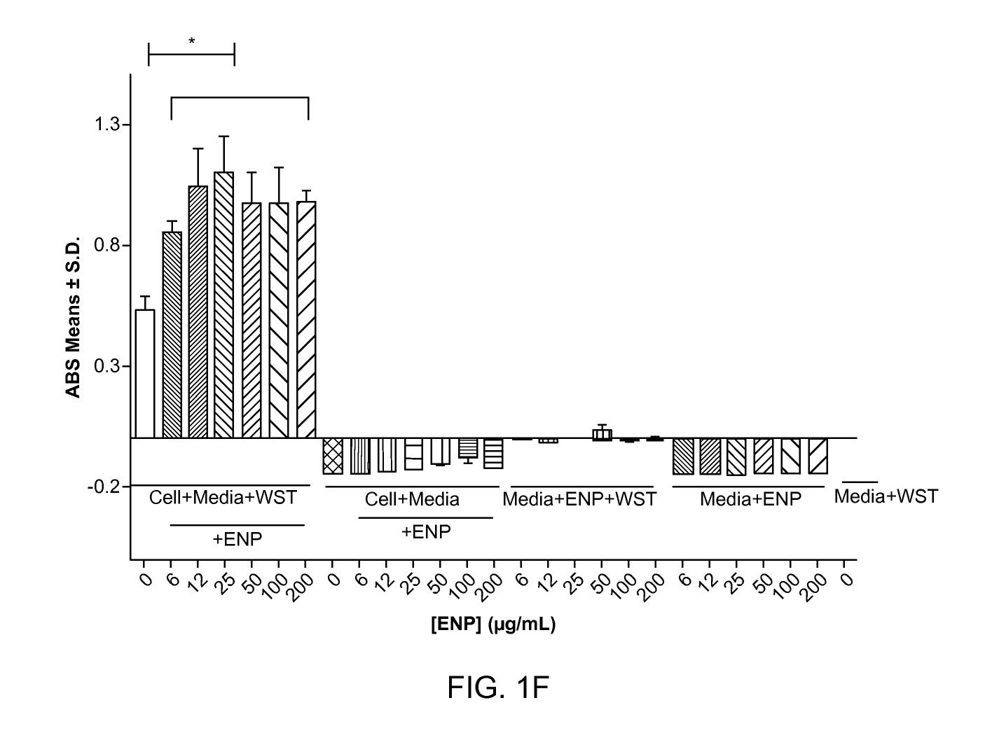

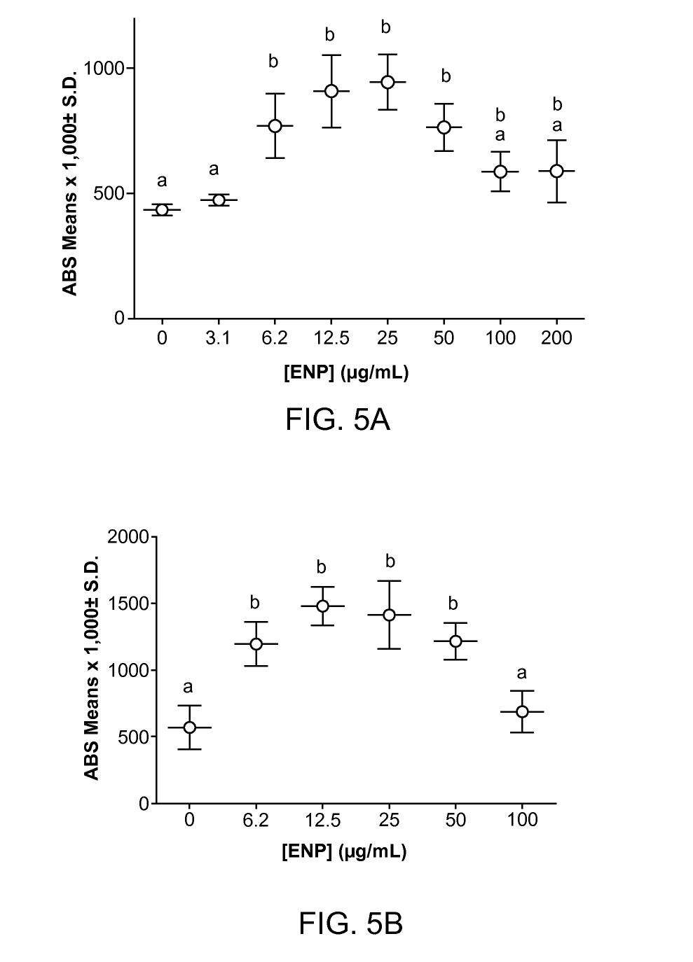

Human mammary epithelial cells (HMEC 184) (˜40% confluent) exposed to ENPs from 3.1 to 200 μg/mL for 24, 48, and 72 h showed a dose-dependent increase in metabolic activity, as measured by the WST-1 assay (FIGS. 1a, 1b, 1c). The increase in WST-1 indicates an increase in the activity of mitochondrial succinate dehydrogenase. Using HMEC 184 confluent cells (˜90% confluent), ENPs induced a dose-dependent increase in metabolic activity. Two human epithelial breast cancer cell lines (MCF-7 and MDA-MB-231) grown in different media were also incubated with varying doses of ENPs for 24 h; again, results showed a dose-dependent increase in metabolic activity (FIGS. 1d, 1e). In a separate experiment, ENPs were mixed with culture media (3.1 to 200 μg/mL) and added to 96-well plates, either 24 h before seeding the HMEC 184 cells or at the same time as cell seeding, and a WST-1 assay was performed 2 days later. A similar trend of dose-dependent increased metabolic activity was seen in both cases (FIG. 5). Because a false-negative toxicity has been reported previously with MTT-formazan in interacting with NPs, but not with the WST-1 assay (Worle-Knirsch et al., 2006), additional control cells were subjected to WST-1 to rule out the possibility of reagent interaction with ENPs (FIG. 1f).

FIG. 1. Metabolic activity of HMEC 184 cells (a, b, c), MDA-MB-231 (d), and MCF-7 (e) following 24 (a, d, e), 48 (b), and 72 h (c) exposure to various doses of ENPs (μg/mL). Different controls were tested in HMEC 184 cells to examine the effect of ENPs on the accuracy of the WST-1 assay (f). Data are means±SD. Groups not sharing the same letter are different at the 95% level according to ANOVA analysis (p<0.0001, Tukey's honest significant difference). * Significantly different (comparing all groups vs. control, Dunnett's post test).

FIG. 5. Metabolic activity of HMEC 184 cells following 48 h exposure to ENPs. ENPs (μg/mL) were pre-incubated for 24 h in plates with culture media at different concentrations before cells were seeded and incubated for 48 h (a). Cells were incubated for 48 h with ENPs (μg/mL) at different concentrations at the same time as cell seeding (b). Groups not sharing the same letter are different at the 95% level according to ANOVA analysis (p<0.0001, Tukey's honest significant difference).

### Dose-Dependent Decrease in Cell Proliferation and Increase in Protein Content of Epithelial Cells Exposed to ENPs

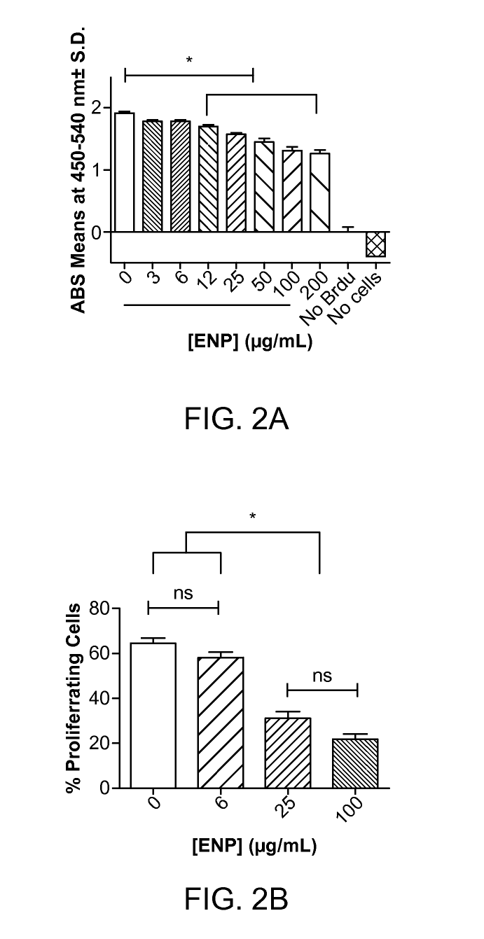

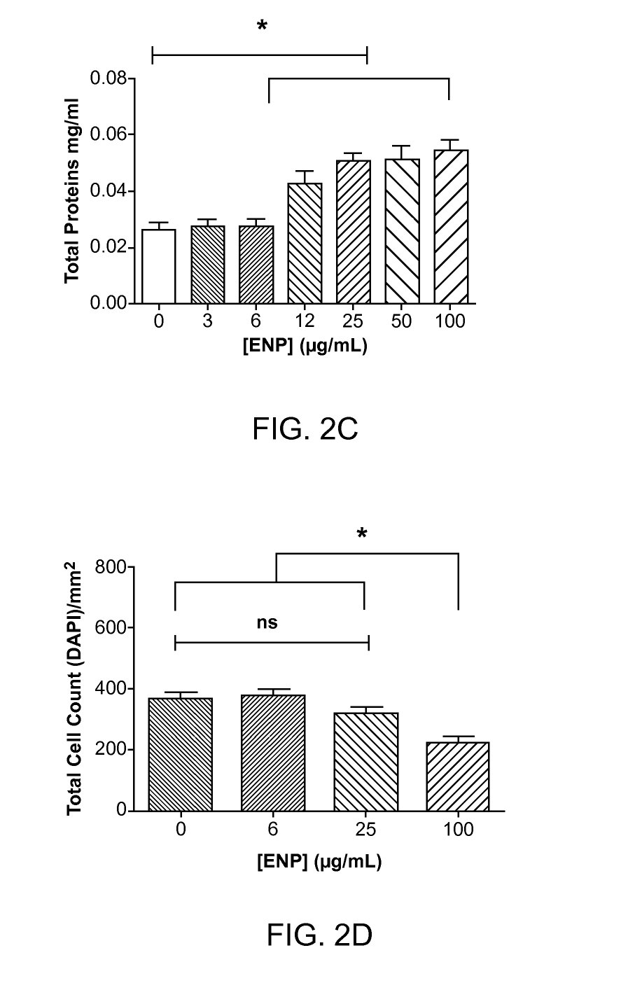

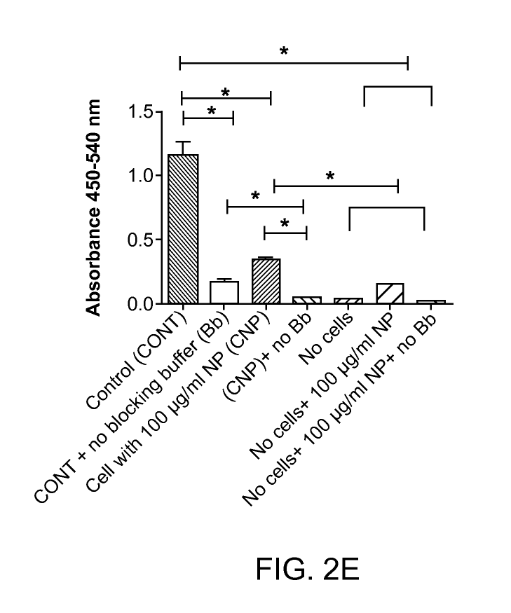

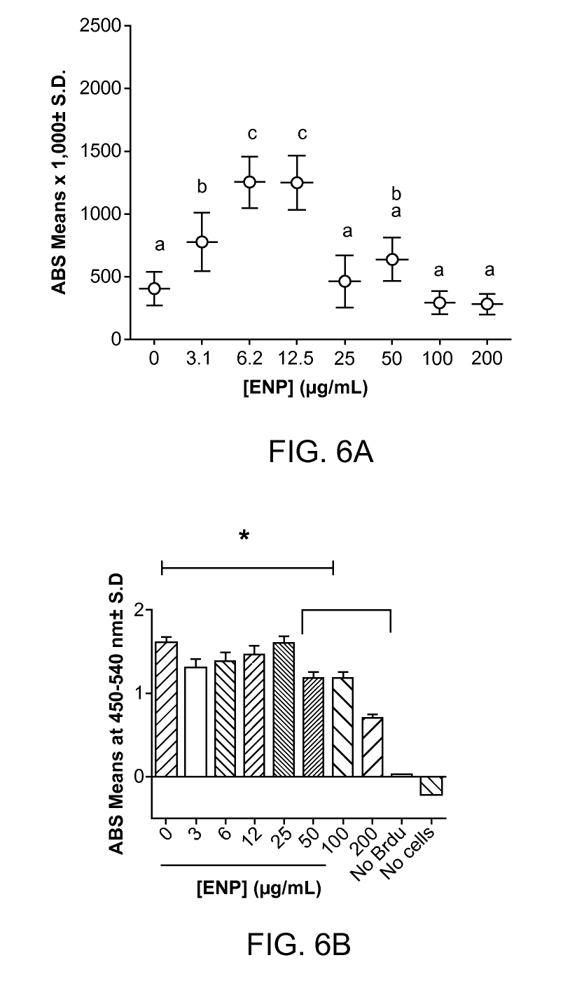

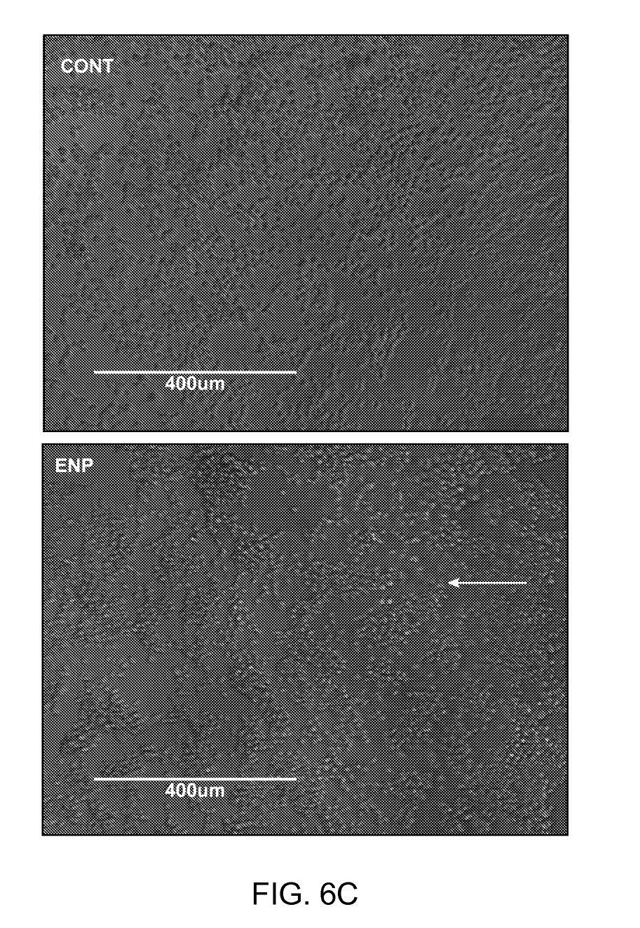

A dose-dependent decrease in cell proliferation, measured with a 5-bromo-2′deoxyuridine (BrdU) ELISA assay, was observed in the HMEC 184 cells after 24 h incubation with ENPs (FIG. 2a). The decrease in proliferation was further confirmed with a proliferation assay that quantified proliferating HMEC 184 cells labeled with 5-ethynyl-2′-deoxyuridine (EdU) using a fluorescent azide reaction (FIG. 2b). An increase in total protein content in the HMEC 184 cells, measured with a BCA assay, was observed after 24 h of incubation with ENPs (FIG. 2c), and a decrease in total cell count was seen only in those cells incubated with a high dose of ENPs (100 μg/mL), as measured with DAPI stain (FIG. 2d). Additional control cells were subjected to BrdU assays to rule out the possibility of reagent interaction with ENPs (FIG. 2e). Neural progenitor cells (NPC) were treated with 0 to 200 μg/mL ENPs to test whether results were specific to epithelial cells. A similar dose-dependent increase in metabolic activity and dose-dependent decrease in cell proliferation were seen in those cells (FIG. 6).

FIG. 2. Cell Proliferation (a, b), total proteins (c), and cell count (d) in HMEC 184 cells following 24 h exposure to ENPs (μg/mL). Cell proliferation (a) was measured with 5-bromo-2′-deoxyuridine (BrdU) incorporated into cellular DNA for 6 h, and a BrdU mouse mAb used to detect the BrdU-labeled DNA. A 5-ethynyl-2′-deoxyuridine (EdU) cell proliferation assay (b) demonstrated that ENPs decrease the percent of proliferation of HMEC 184 cells in culture in a dose-dependent manner. Different controls were tested in HMEC 184 cells to examine the effect of ENPs or blocking buffer on the accuracy of the BrdU assay (e). Data are means±SD. * Significantly different at the 95% level according to ANOVA analysis (comparing all groups vs. control, Dunnett's post test).

FIG. 6. Metabolic activity (a) and cell proliferation (b) in neural progenitor cells (NPC) following 24 h exposure to ENPs (μg/mL). Metabolic activity and cell proliferation were measured with WST-1 and BrdU assays. A dose-dependent increase in metabolic activity and dose-dependent decrease in cell proliferation were seen in this cell line. ENPs formed a visible network with NPC media that adhered to cells in culture (c).

ENPs Formed a Visible Network with Serum Proteins that Adhered to Cells in Culture. ENPs Entered the Cells and Caused an Increase in Total Mitochondrial Volume Without an Increase in Mitochondrial Biogenesis

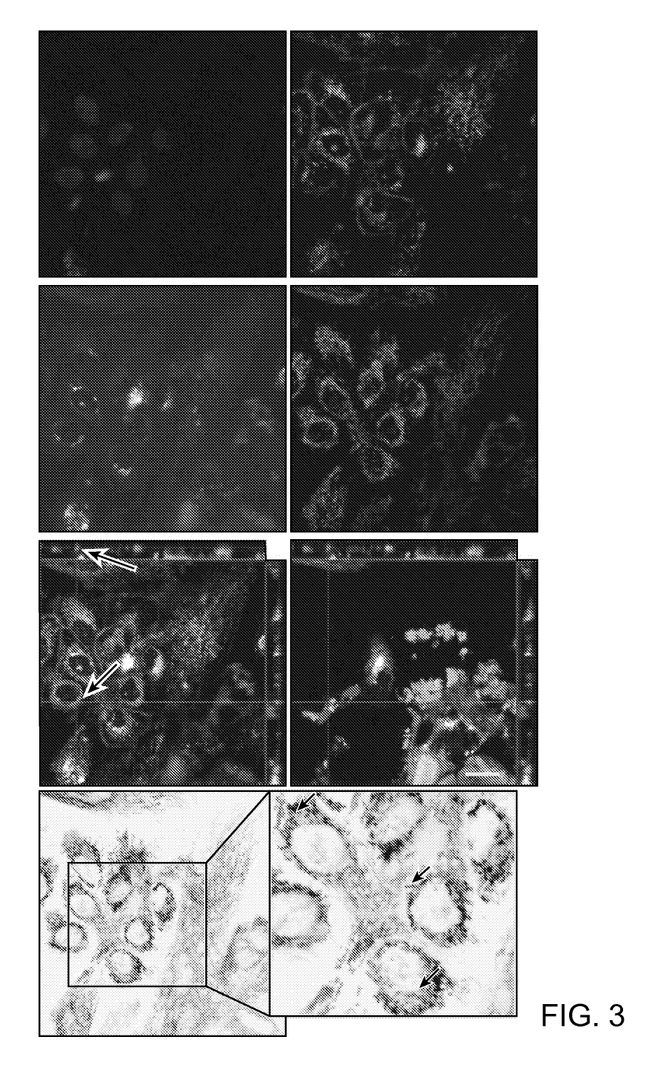

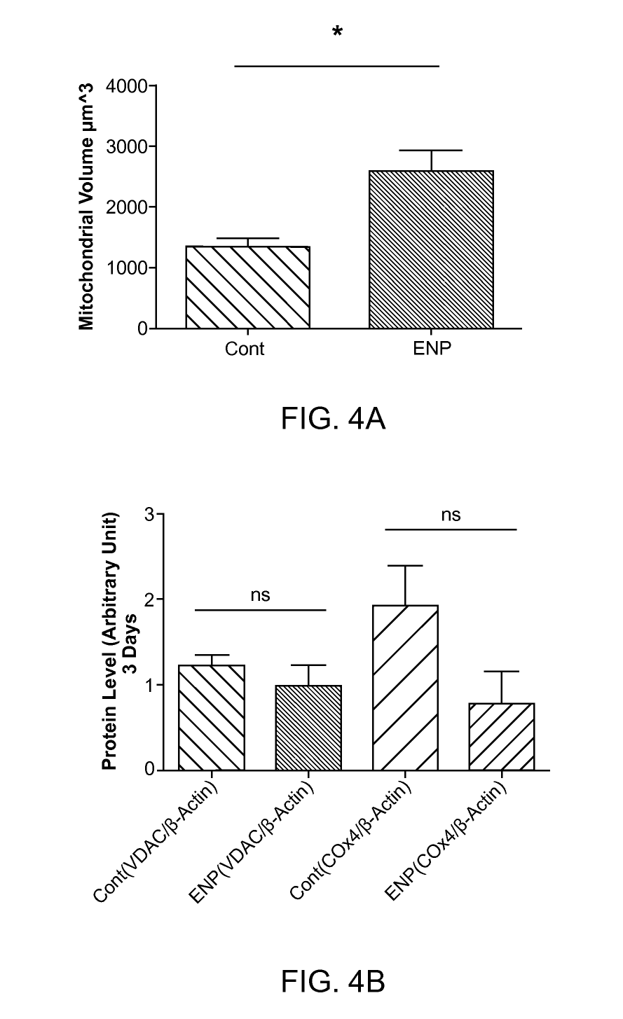

An opalescent flocculate was visible when ENPs were added to HMEC 184, MDA-MB-231, and MCF-7 culture media. No flocculate was observed with the addition of ENPs to serum-free medium or PBS. Labeling of ENPs with Nile red fluorescent congregated dye and examination with confocal microscopy showed that ENPs entered the cells (FIG. 3), but the majority of ENPs aggregated into clumps with proteins that formed a clearly visible network closely attached to cells. Colocalization analysis in ImageJ showed that some ENPs are localized with mitochondria (FIG. 3g). No fragmentation was seen in mitochondrial networks in HMEC 184, MDA-MB-231, and MCF-7 cells treated with ENPs at 25 μg/mL for 24 and 72 h. There was an increase in the total mitochondrial volume in HMEC 184 cells treated with 25 μg/mL ENPs, when compared to untreated control cells as measured with Imaris software (FIG. 4a). However, a decrease in Cox4 and VDAC proteins was seen with Western blotting, but neither was a significant change (FIG. 4b). This implies an increase in cell size without a corresponding increase in mitochondrial biogenesis.

FIG. 3. HMEC 184 cells after 3-day exposure to Nile red-ENP (25 μg/mL) as observed with a Zeiss LSM 780 confocal microscope. Nuclei were stained with 33342 Hoechst dye (a), membranes were stained with wheat germ agglutinin (b), ENPs were conjugated to Nile red (c), and mitochondria were stained with MitoTracker (d). Ortho-view of the z-stack shows that some ENPs are inside the cells (e) and some are aggregated on top of the cells (f). Colocalization analysis in ImageJ shows that some ENPs are colocalized with mitochondria (purple dot) (g). Whole images were contrastenhanced using ImageJ software. Scale bar=20 μm.

FIG. 4. Mitochondrial volume (a) and protein content (relative to β-actin) (b) in HMEC 184 cells after 3-day exposure to ENPs (25 μg/mL). Mitochondria were stained with MitoTracker, and mitochondrial volumes (μm̂3) were measured with Imaris software. There was an increase in mitochondrial volume in cells treated with ENPs as compared to control cells. A decrease in Cox4 and VDAC proteins was seen with Western blotting, but neither was a significant change. Data are means±SD. *Significantly different at the 95% level according to ANOVA analysis (comparing all groups vs. control, Dunnett's post test).

Proteomic Mass Spectrometry Showed that the ENP-Serum Protein Network Contains Proteins Sharing Common Interpro Domains and Exhibiting Protease, Antiprotease, Epidermal Growth Factor, Adhesion, and Binding Properties

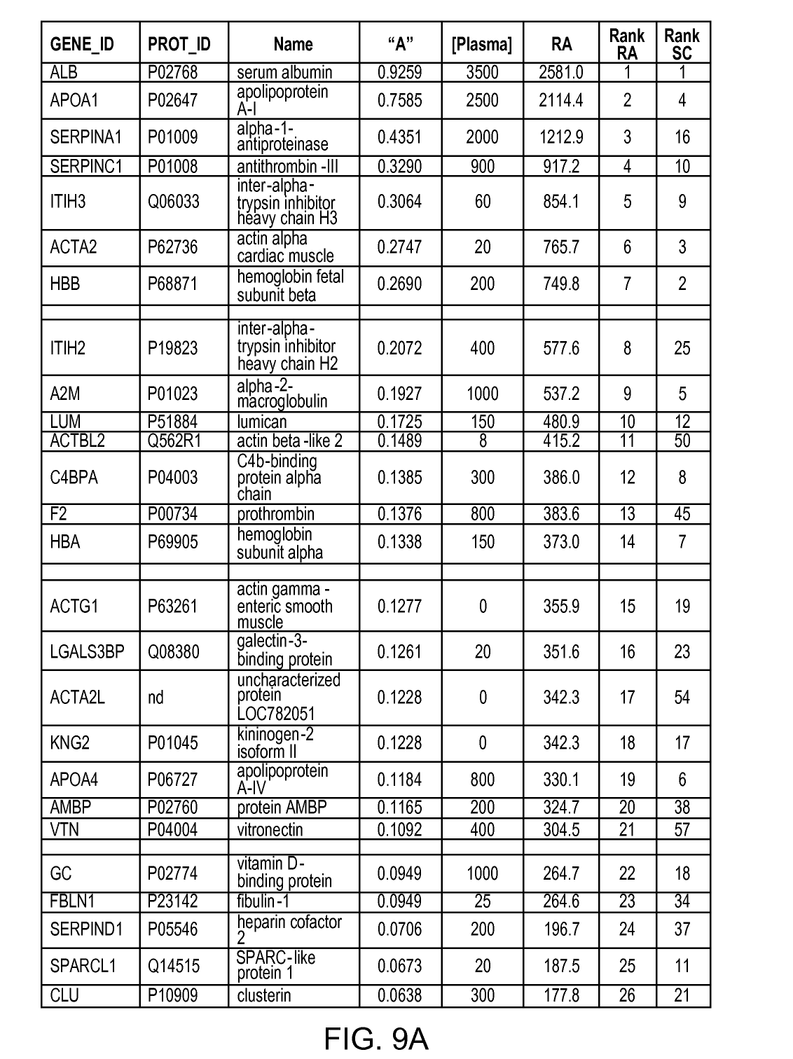

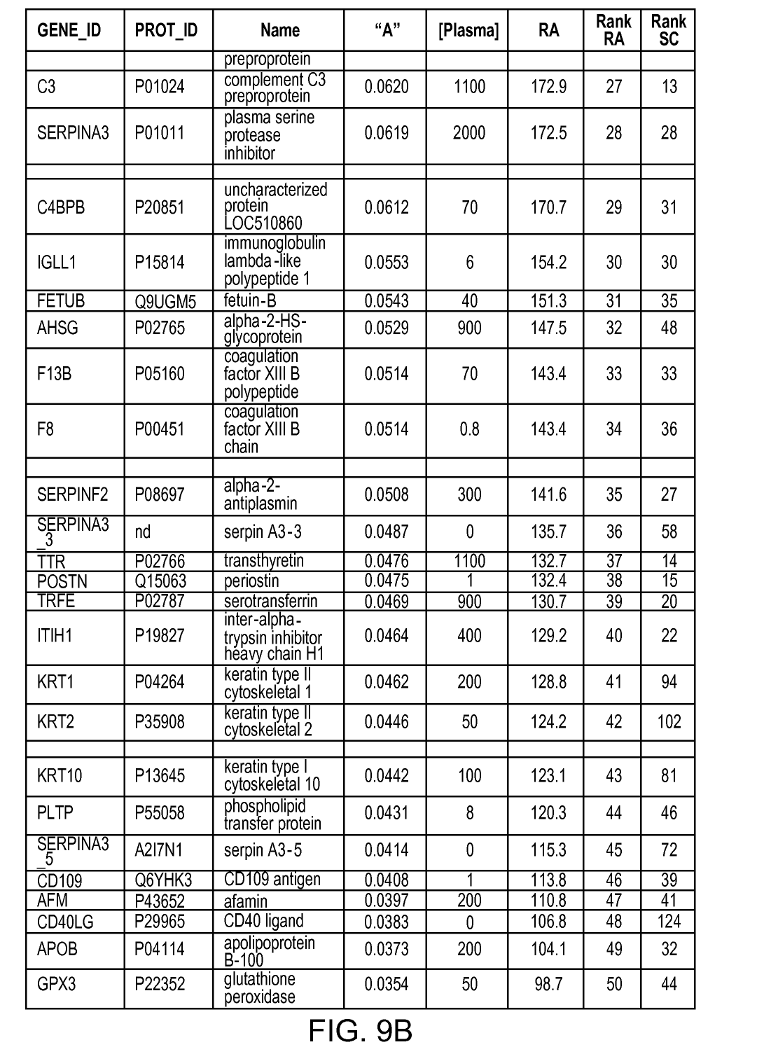

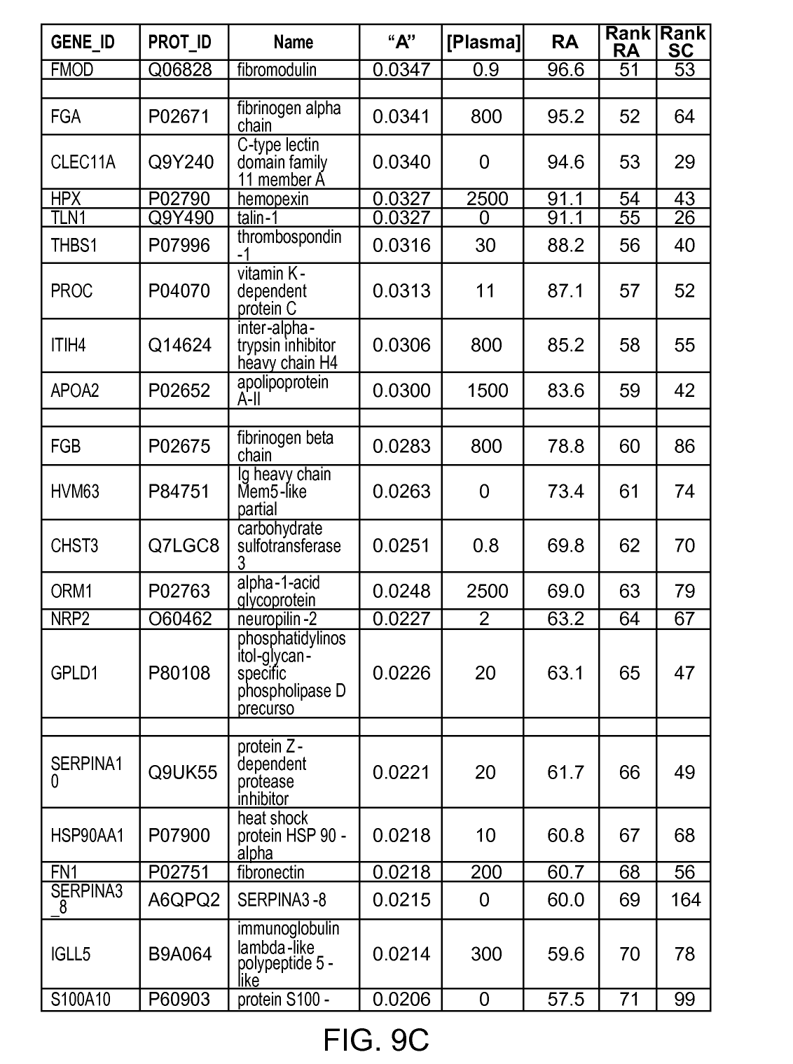

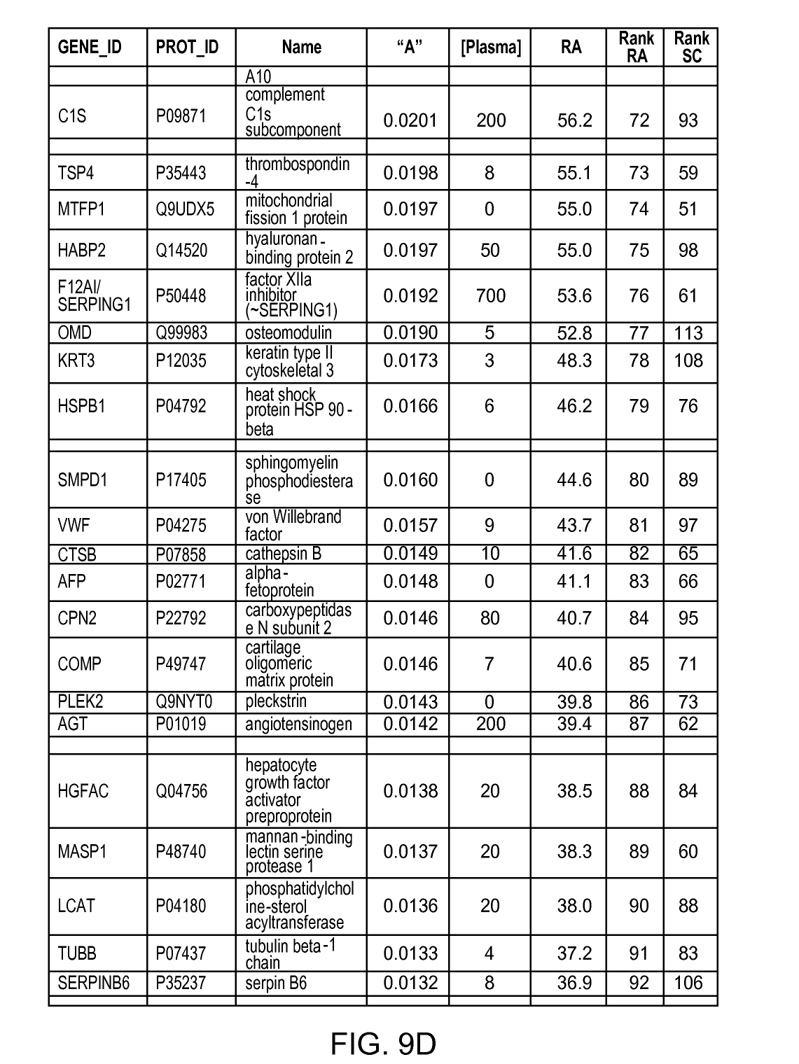

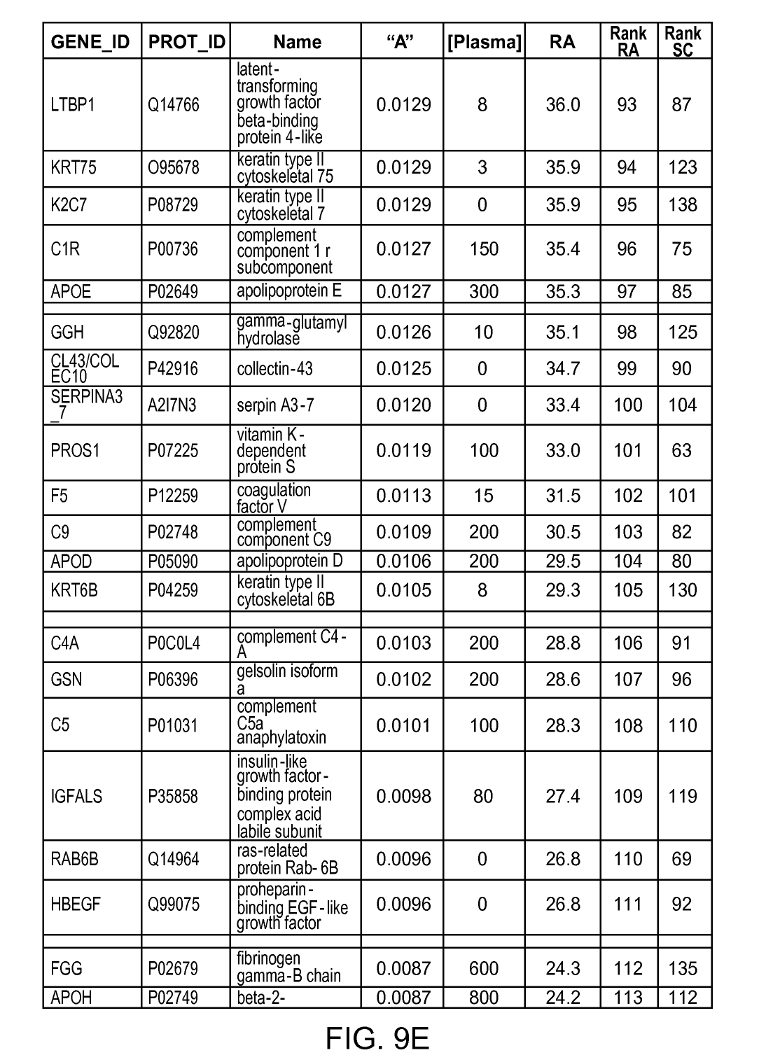

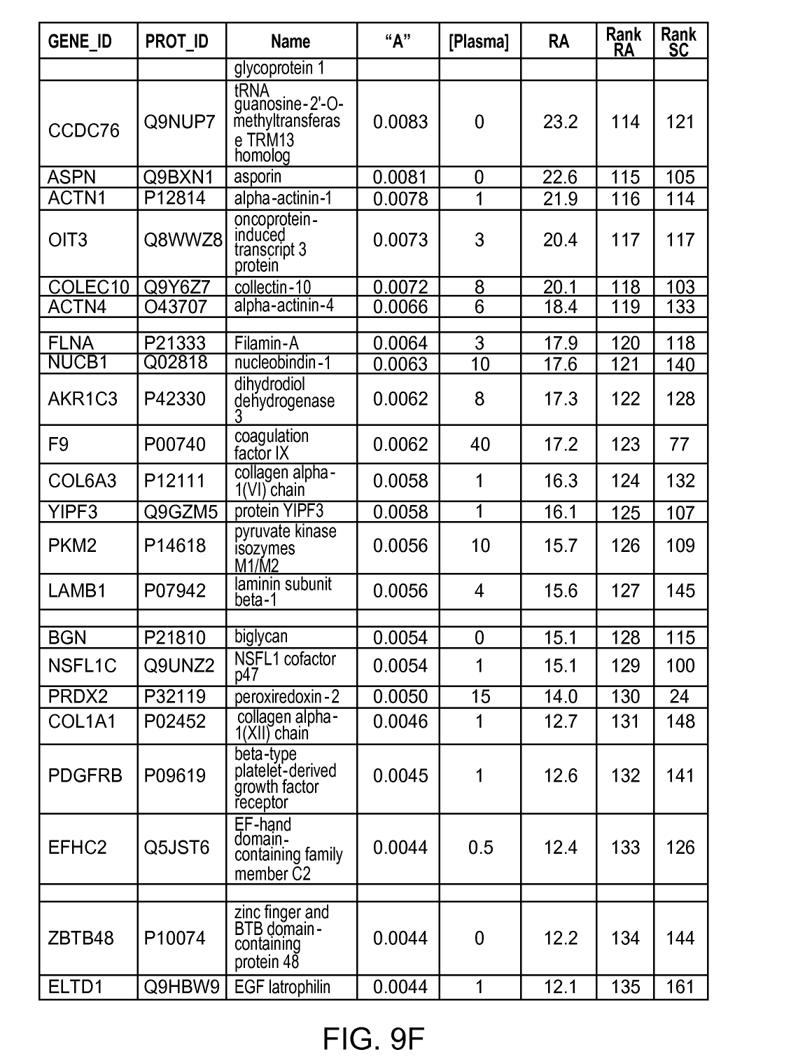

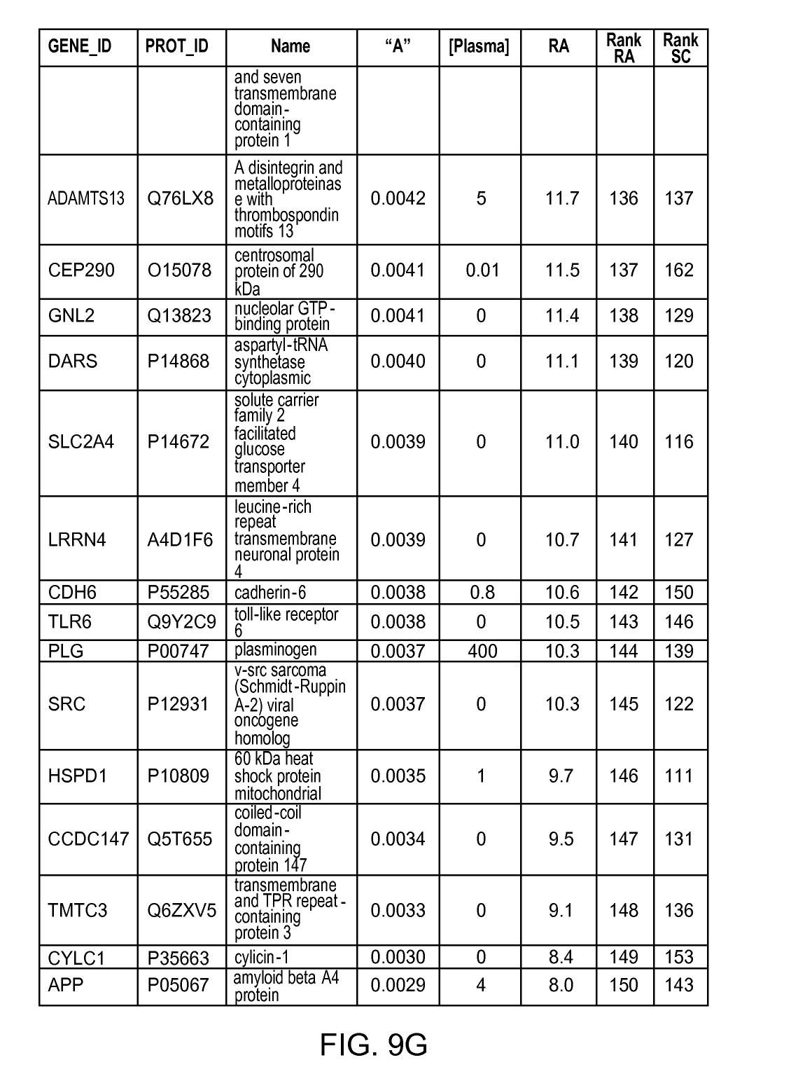

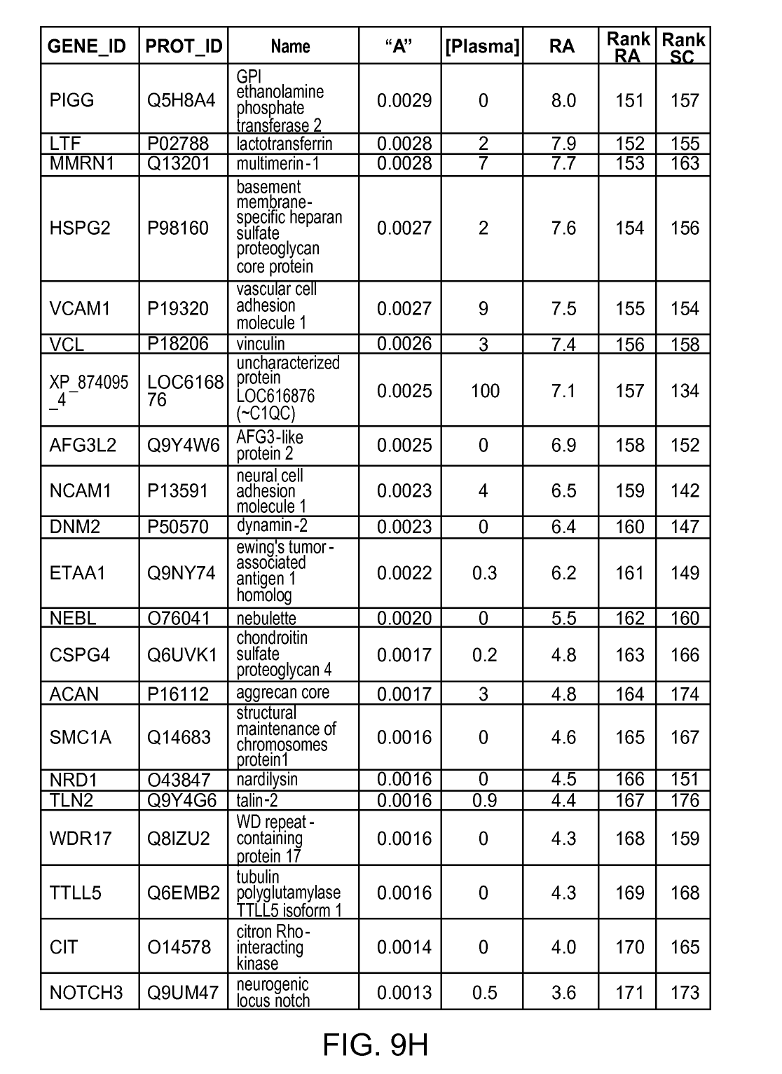

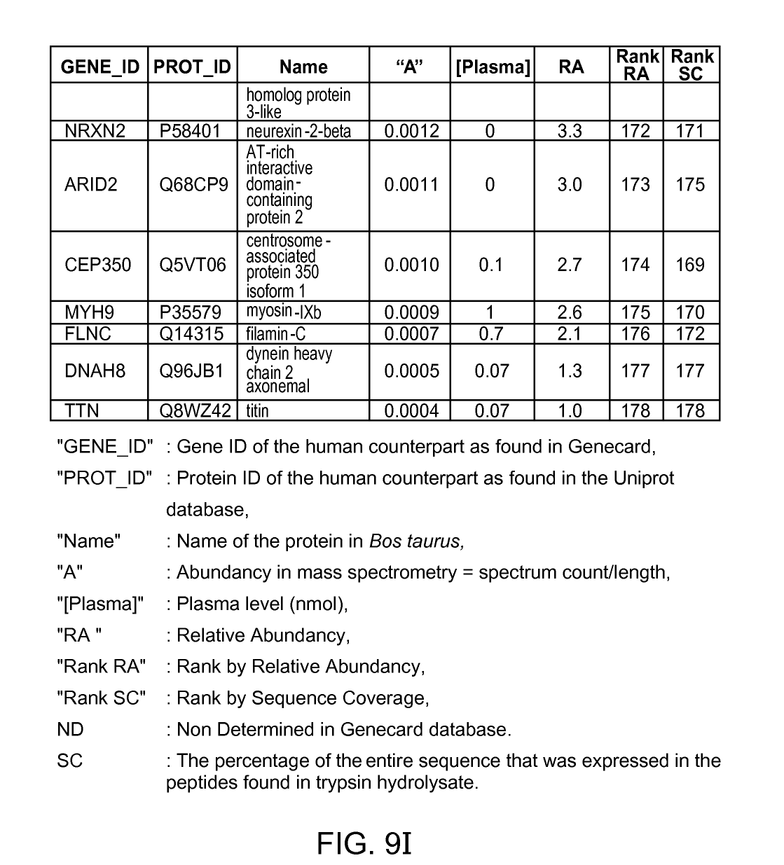

To identify the nature of the visible flocculate, the pellet generated from centrifugation of a mixture of serum and ENPs was washed twice with PBS and exposed to organic extraction. No lipids were seen in the organic phase on TLC, but the aqueous phase showed a peak at 280 nm and an increased absorbance at 240 nm, with the UV/VIS spectrum suggesting the presence of proteins. The pellet was examined with SDS-PAGE and MALDI-TOF mass spectrometry, which identified 290 non-redundant proteins belonging to Bos taurus and 9 human cytokeratins. From MALDI-TOF MS, 178 proteins were identified and analyzed (FIG. 9). Sequence coverage varied from 79.2% for albumin to 0.4% for titin. The relative abundances (RAs) were calculated as the ratio of the most abundant to the least abundant protein, varied between 2,581 and 1.

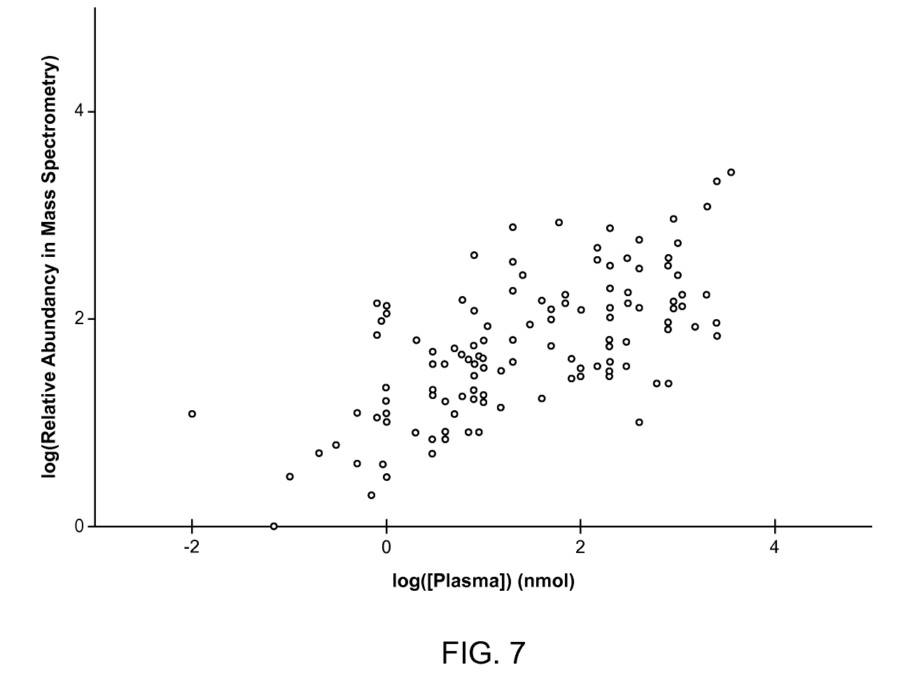

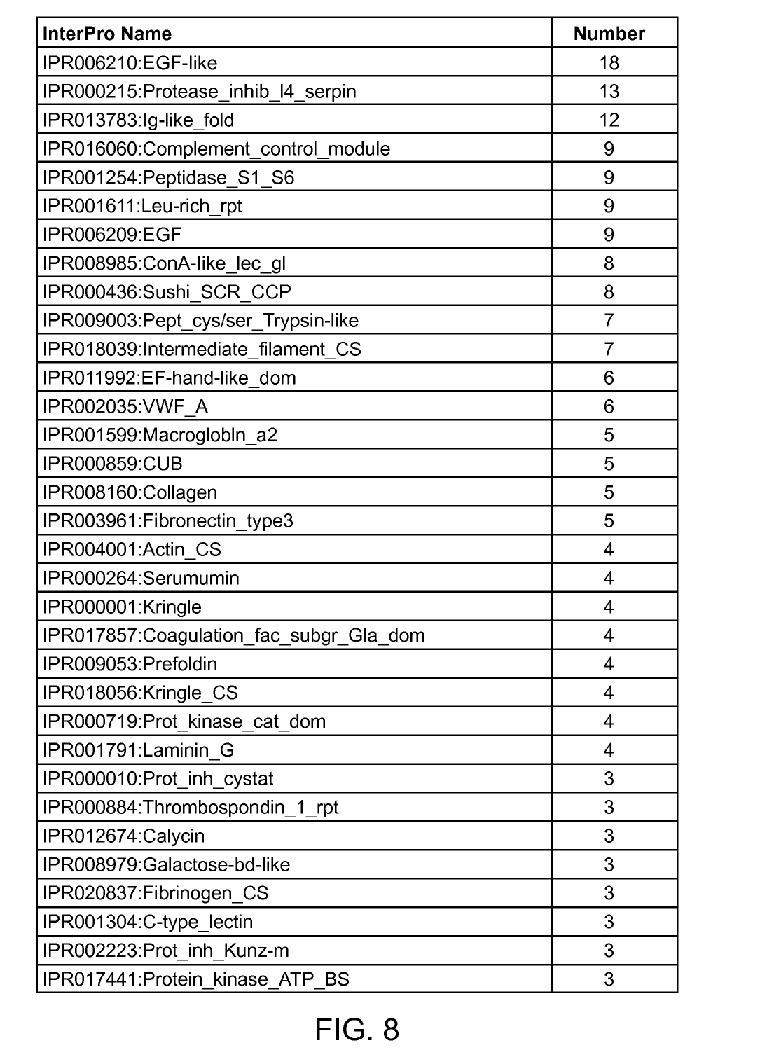

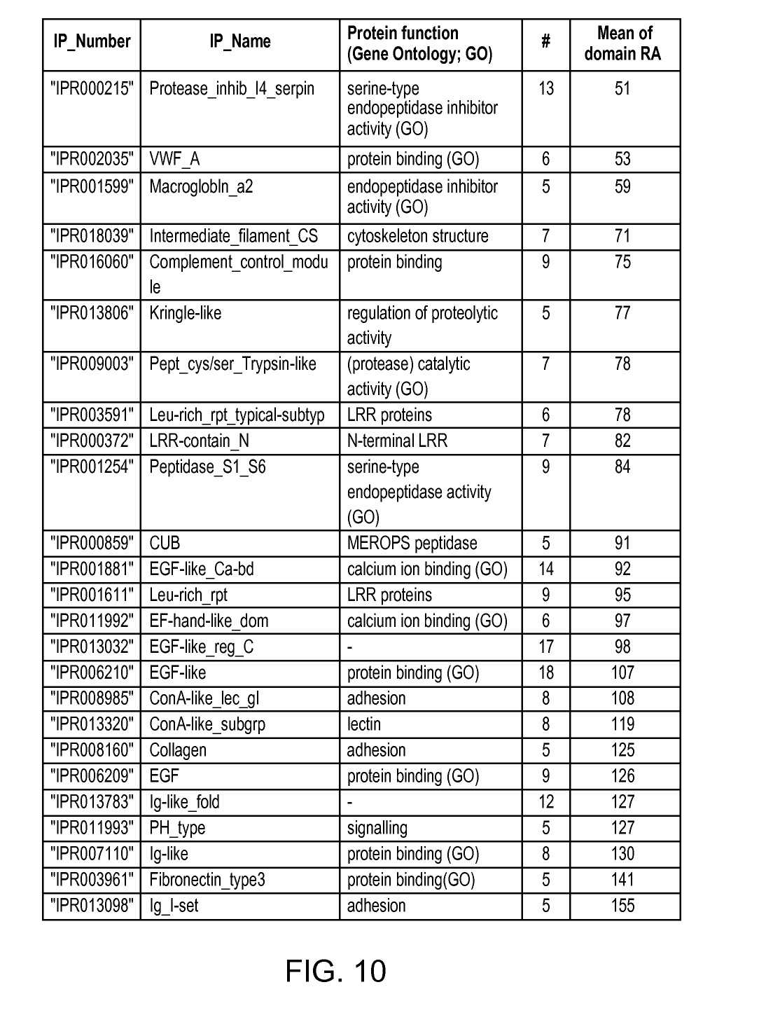

Regression analysis at a 95% confidence level showed a linear correlation between protein abundance in mass spectrometry and the protein concentration in plasma (FIG. 7). However, notable exceptions were seen later in the InterPro analysis. Because the coefficient of correlation (r) was only 0.66, and because some proteins were present at concentrations less than 1 nmol in plasma (e.g., kininogen-2 isoform II, actins, periostin, and serpinA 3-3), but were abundant in the mass spectrometry chromatograms (FIG. 9), we believe that proteins were not randomly fixed on ENPs according to their abundance in FBS. Therefore, we retrieved the InterPro domains of most of the 178 proteins we analyzed. The most frequently shared InterPro domain was “IPR006210:EGF-like,” shared by 18 different proteins (FIG. 8). Approximately 120 of 170 analyzed proteins (70%) shared at least 2 common domains. A high number of proteins belonging to InterPro domains are known to be involved in endopeptidase inhibitor activity, proteolytic activity or its regulation, protein binding, calcium binding, cell adhesion, and signaling, or have certain structural motifs like leucine-rich or immunoglobulin domains (FIG. 8). By calculating the mean of relative abundance at the domain level, but not at the entire protein level, we found that proteins were coated not only due to their relative amounts in FBS, but also due to their affinities for ENPs, reflecting their composition in domains rather than the structure of the entire protein (FIG. 10). When submitting 178 genes to the STRING database, 169 protein encoding genes were recognized, and more than 80% of these showed interactions, either at evidence, confidence, or action levels. Seemingly, the purified proteins were also isolated by interactions between and among themselves (e.g., by protease inhibitor/protease interactions, or known binding properties of individual proteins). Serpins, proteases, and coagulation factors were found at the central core around which proteins involved in cell adhesion, growth, differentiation, and migration clustered.

FIG. 7. Linear regression analysis between the logarithm of plasma concentration (nmol) and the logarithm of relative abundance as obtained by mass spectrometry data. Regression analysis at a 95% confidence level showed a linear correlation between protein abundance in mass spectrometry and the concentration in plasma (r=0.66, p<0.0001).

FIG. 8. Commonly shared domains of proteins purified by ENPs. The InterPro domains were retrieved from “www” followed by “.ebi.ac.uk” followed by “/interpro/”. About 120 of 170 analyzed proteins (70%) shared at least 2 domains.

FIG. 9. A list of proteins from FBS purified by ENPs and identified with MALDI-TOF mass spectrometry. Mass spectrometry analysis identified ˜299 proteins from FBS that were attached to ENPs. Of these, 178 proteins are listed here and were identified and analyzed for: name of product in Bos taurus (as appeared in Unigene), name of gene in Homo sapiens counterpart, name of human counterpart protein, and levels in human plasma. A notable fraction of those proteins (˜45, highlighted in yellow) were either never described in plasma or are present in plasma in pathological conditions. Proteins are ranked according to their abundance (RA) and sequence coverage (SC). Sequence coverage varied from 79.2% for albumin to 0.4% for titin.
“GENE_ID” : Gene ID of the human counterpart as found in Genecard,
“PROT_ID” : Protein ID of the human counterpart as found in the Uniprot database,
“Name” : Name of the protein in Bos taurus,
“A”: Abundancy in mass spectrometry=spectrum count/length,
“[Plasma]” : Plasma level (nmol),
“RA ” : Relative Abundancy,
“Rank RA” : Rank by Relative Abundancy,
“Rank SC” : Rank by Sequence Coverage,
ND: Non Determined in Genecard database.
SC: The percentage of the entire sequence that was expressed in the peptides found in trypsin hydrolysate.

FIG. 10. Relative abundance at the domain level. A list of selected frequently appearing InterPro domains (IP number & IP name), their function as accepted and annotated by Gene Ontology, the number of different proteins sharing them, and their mean of relative abundance.

Microarray Analysis for HMEC 184 Cells Treated with 25 μG/Ml ENPs Showed Activation of Proliferation and Growth Pathways

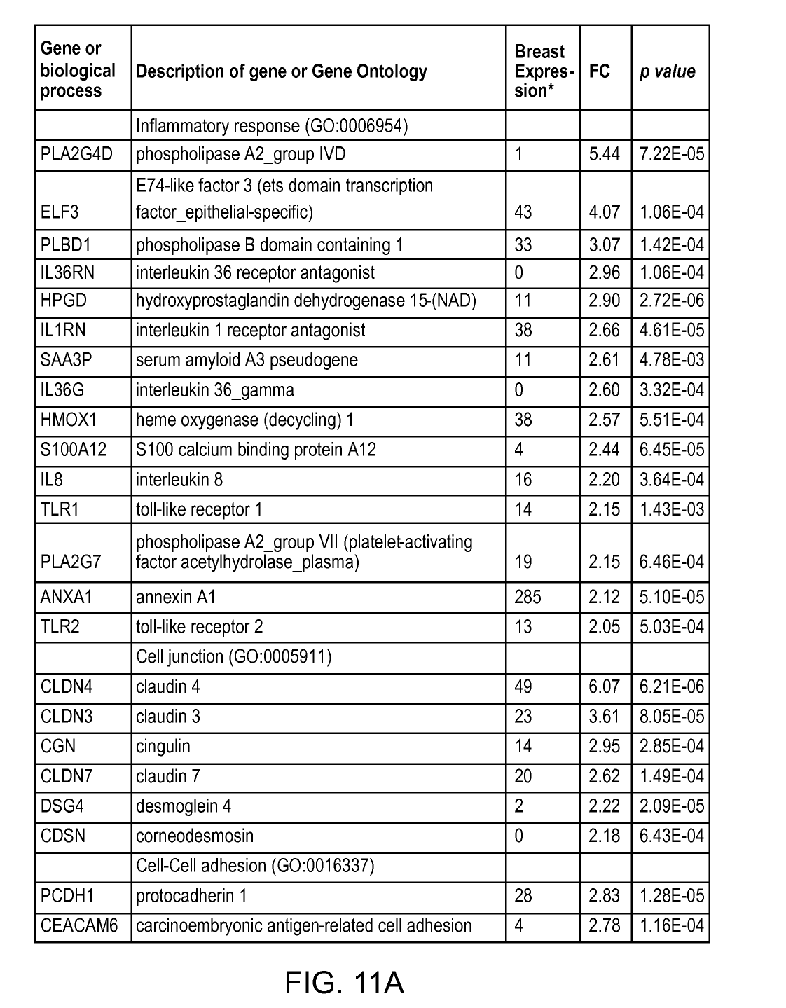

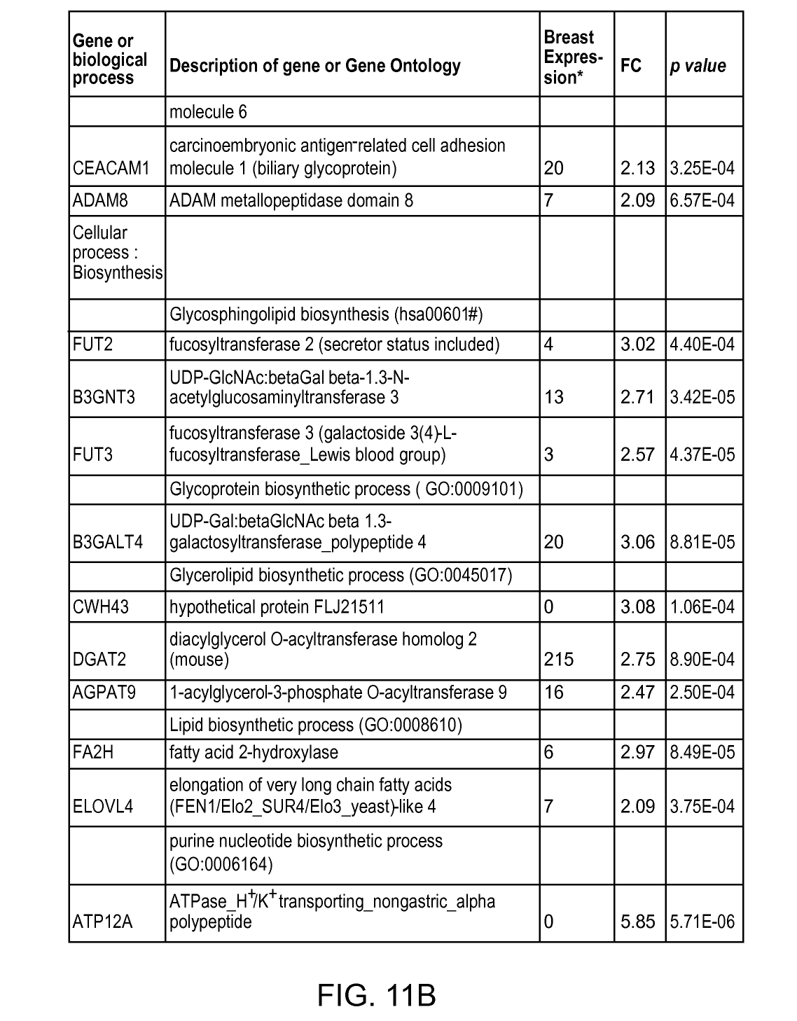

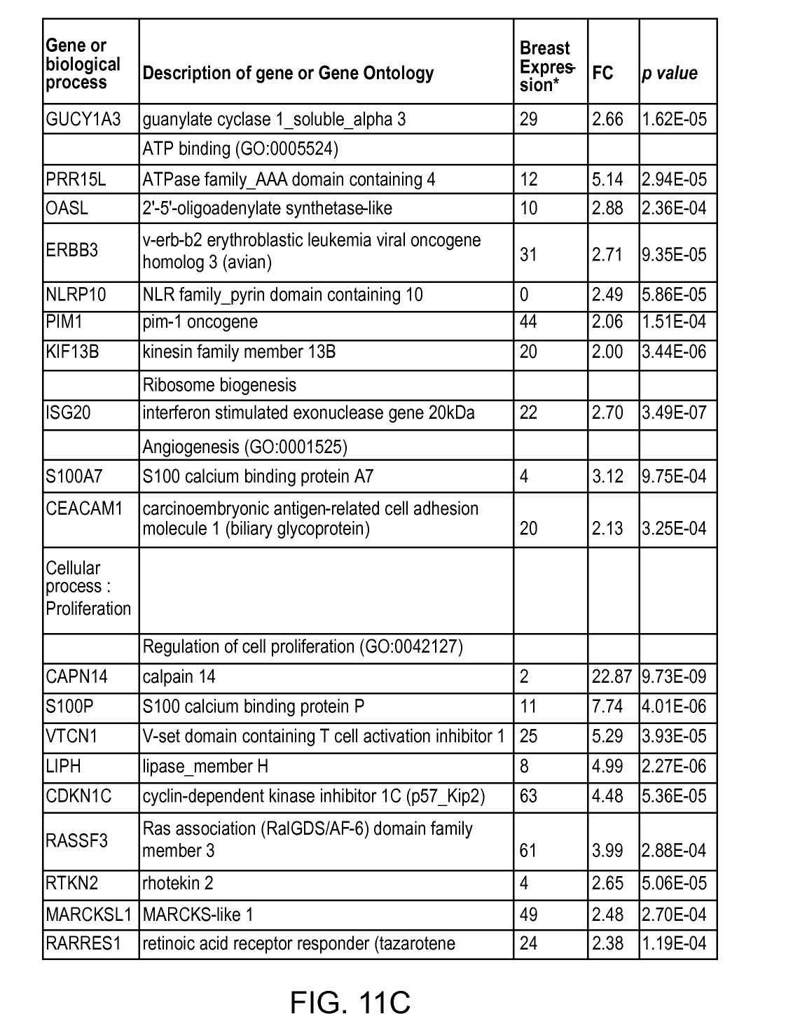

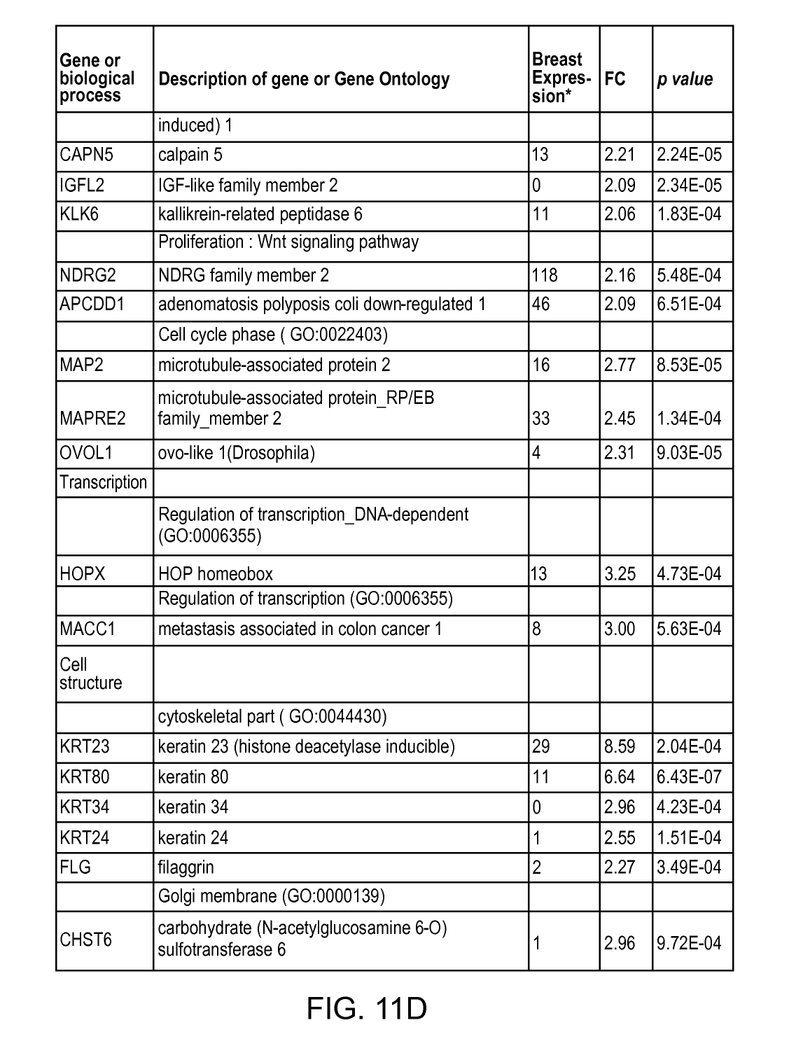

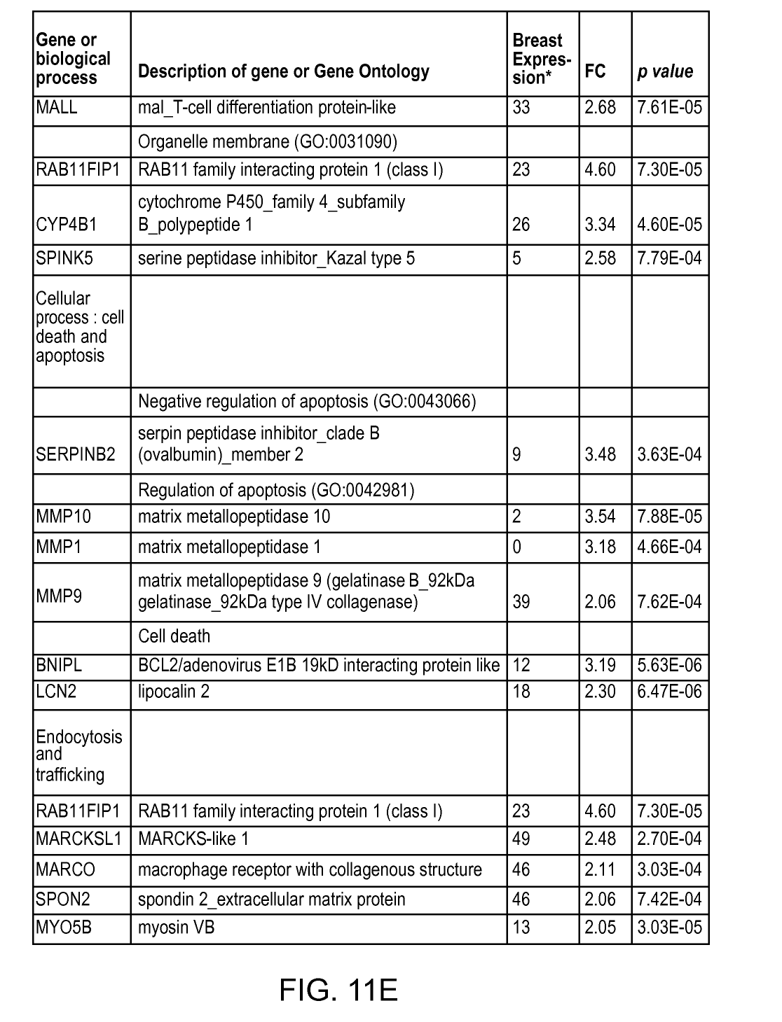

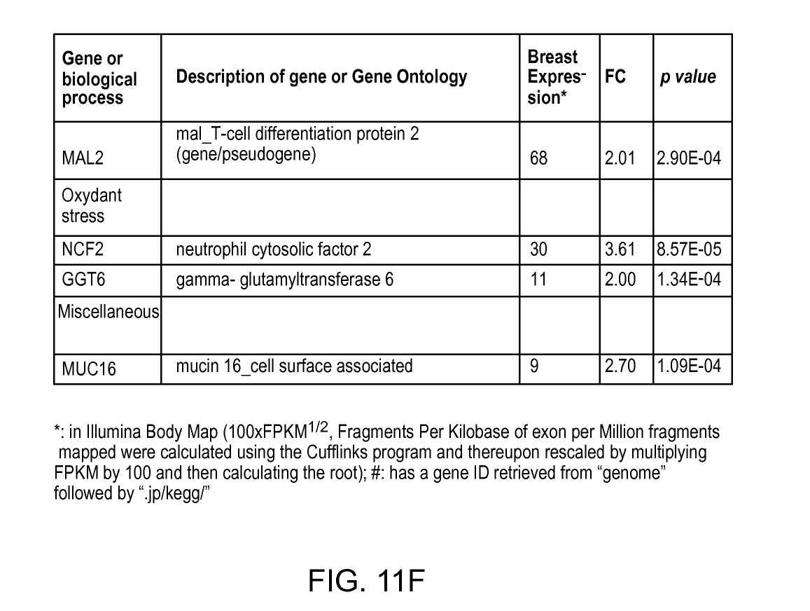

Microarray analysis was performed on HMEC 184 treated cells to identify the pathways induced by ENP treatment. HMEC 184 treated cells indicated 38 and 287 genes (50% and 90% confluence, respectively) whose expression was significantly altered when compared to control. The stringency of transcriptomic analysis was higher in the 90% confluence series (p<0.001) than the 50% confluence series (p<0.05). Few genes were downregulated in either cell-culture condition: 3 and 4 genes for the 50 and 90% confluence series, respectively (FIG. 11).

FIG. 11. List of significantly upregulated genes in HMEC 184 cells (90% confluence) following 24 h exposure to ENPs (25 μg/mL) identified with DAVID Genecard DB. Gene, description, breast expression, fold change as compared to control (FC), and p-value. *: in Illumina Body Map (100×FPKM^{1/2}, Fragments Per Kilobase of exon per Million fragments mapped were calculated using the Cufflinks program and thereupon rescaled by multiplying FPKM by 100 and then calculating the root); #: has a gene ID retrieved from “genome.jp/” followed by “kegg/”.

Several of the up-regulated genes were genes involved in pathways of proliferation, growth, and transformation. These genes included PIM-1, which contributes to cell proliferation and survival; VTCN1, which promotes epithelial cell transformation; ADRA1B (up-regulated in the 50% confluence dishes), which activates mitogenic responses and has been found in normal and cancerous breast cell lines; LCN2, described as a gene involved in breast tumor progression; ELF3, an ETS domain transcription factor that is epithelial-specific and is known to transactivate alone, or synergistically with other genes also upregulated in our experiment (such as CLND7,FLG, KRT8, SPRR1A, MMP1, MM9, and TGM3), epithelial cell differentiation; and NDRG2, which is involved in WNT signaling pathway.

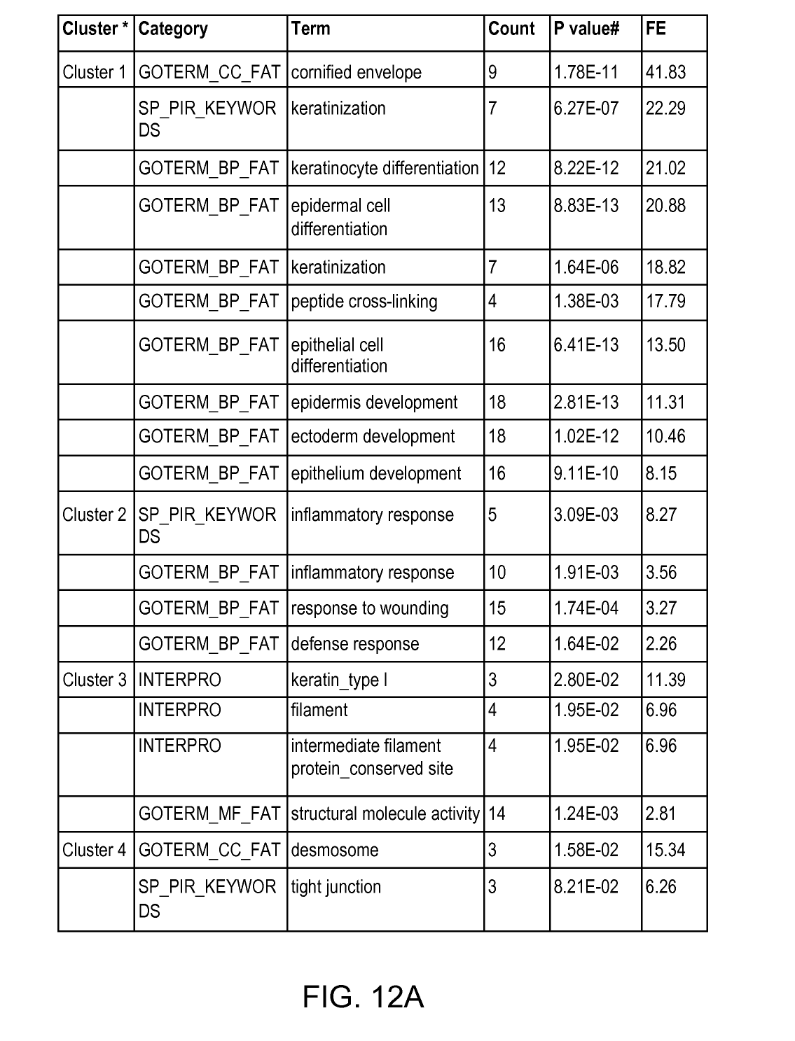

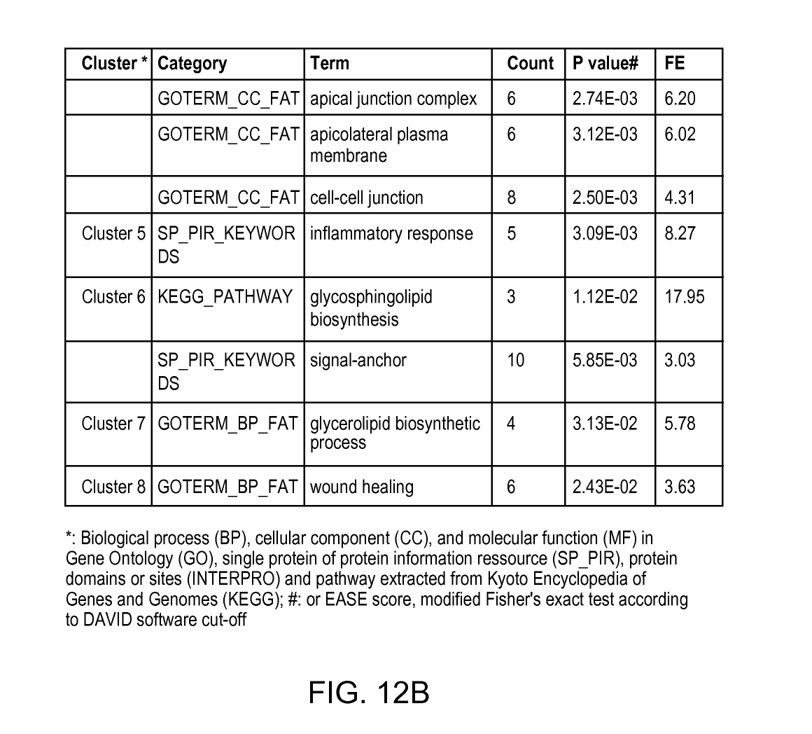

The functional annotation using DAVID revealed GO terms such as epithelial cell differentiation, epidermis development, response to wounding, and ectoderm development with a highly significant probability (p<10E-12, FC>10). These GO terms indicate that ENP action caused a spatial organization of breast epithelium (FIG. 12). The effect of ENP on epithelial cell organization was also seen in significant changes within genes involved in apicolateral plasma membrane organization, cell-cell junction, cell-cell adhesion, and apical junction complex organization. These genes included CLDN4, CLDN3, CGN, CLDN7, DSG4, and CDSN (FIG. 11).

FIG. 12. Functional annotation analysis of microarry data sets using DAVID. GO terms were significantly enriched in genes at least twofold upregulated in HMEC 184 cells (90% confluence) in response to 24 h exposure to 25 μg/mL ENPs. *: Biological process (BP), cellular component (CC), and molecular function (MF) in Gene Ontology (GO), single protein of protein information resource (SP_PIR), protein domains or sites (INTERPRO) and pathway extracted from Kyoto Encyclopedia of Genes and Genomes (KEGG); #: or EASE score, modified Fisher's exact test according to DAVID software cut-off.

Differentially expressed genes at a level of p<10E-03 were detected in breast tissues by Illumina Body Map, indicating a strong relationship in expression pattern between HMEC 184 cell line and human breast tissue. Only 9 of 84 genes (˜11%) displayed no expression in human breast tissue: IL36RN, IL36G, CDSN, CWH43, ATP12A, NLRP10, IGFL2, KRT34, and MMP1. Eighteen of the upregulated genes were described as upregulated genes in epidermal barrier function: A2ML1, ADAM8, BNIPL, CDSN, CLDN3, CLDN4, CLDN7, DSG4, FLG, IGFL2, KLK6, KRT23, KRT24, KRT34, KRT80, LIPH, SERPINB2, and SPRR1A.

Protein/protein interactions of 84 overexpressed genes in HMEC 184 cells, as retrieved from String database, were visualized. HMEC 184 cells (90% confluence) were incubated with and without 25 μg/ml ENPs for 24 h. Total RNA was extracted and analyzed with microarray. There were 287 genes whose expression was significantly altered when compared to control. Forty-one of the 84 genes in the 90% confluence series recognized by the String database (“string-” followed by “db.org”) were linked at confidence, evidence or action level. Regarding the action level, some relevant catalysis involve: (i) initial activation of proMMP9 by MMP1, (ii) proMMP-9 activation by MMP-10, (iii) CEACAM1&6 heterodimer, (iv) MMP9 potentializing IL8, and (v) complex-forming of serum amyloid A protein with upregulated TLR genes, SAA1/TLR2/TLR1.

### REFERENCES

EIDI, H., JOUBERT, O., ATTIK, G., DUVAL, R. E., BOTTIN, M. C., HAMOUIA, A., MAINCENT, P. & RIHN, B. H. (2010). Cytotoxicity assessment of heparin nanoparticles in NR8383 macrophages. Int J Pharm 396, 156-165.
EIDI, H., JOUBERT, O., NEMOS, C., GRANDEMANGE, S., MOGRABI, B., FOLIGUET, B., TOURNEBIZE, J., MAINCENT, P., LE FAOU, A., ABOUKHAMIS, I. & RIHN, B. H. (2012). Drug delivery by polymeric nanoparticles induces autophagy in macrophages. Int J Pharm 422, 495-503.
HUSSIEN, R. & BROOKS, G. A. (2011). Mitochondrial and plasma membrane lactate transporter and lactate dehydrogenase isoform expression in breast cancer cell lines. Physiol Genomics 43, 255-264.
WORLE-KNIRSCH, J. M., PULSKAMP, K. & KRUG, H. F. (2006). Oops they did it again! Carbon nanotubes hoax scientists in viability assays. Nano Lett 6, 1261-1268.
YOO, J. W., GIRI, N. & LEE, C. H. (2011). pH-sensitive EUDRAGIT nanoparticles for mucosal drug delivery. Int J Pharm 403, 262-267.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for increasing the metabolic activity of a non-phagocytic cell, the method comprising:
culturing the non-phagocytic cell in a culture composition comprising a population of nanoparticles of poly(ethyl acrylate-co-methyl methacrylate-co-trimethylammonioethyl methacrylate chloride) 1:2:0.1 for a period of time sufficient to increase the metabolic activity of the non-phagocytic cell, wherein the average diameter of the nanoparticles is from 30 nm to 100 nm.

2. The method according to claim 1, wherein the culture composition further comprises a growth factor.

3. The method according to claim 1 or claim 2, wherein the culture composition further comprises animal serum.

4. The method according to claim 3, wherein the animal serum is fetal bovine serum.

5. The method according to any of claims 1 to 4, wherein the culture composition comprises conditioned media.

6. The method according to any of claims 1 to 5, wherein the cell is a primary cell.

7. The method according to claim 6, wherein the primary cell is selected from: a primary epithelial cell, and a cancer cell.

8. The method according to claim 7, wherein the primary epithelial cell is a primary human breast cell.

9. The method according to any of claims 1 to 8, wherein the cell is a cancer cell.

10. The method according to claim 9, wherein the cancer cell is a breast cancer cell.

11. The method according to any of claims 1 to 10, wherein the cell is a neural progenitor cell.

12. The method according to any of claims 1 to 11, wherein the average diameter of the nanoparticles of the population is from 55 nm to 90 nm.

13. The method according to any of claims 1 to 11, wherein the average diameter of the nanoparticles of the population is from 60 nm to 80 nm.

14. The method according to any of claims 1 to 13, wherein metabolic activity of the cell is increased relative to a control cell.

15. A method for increasing the metabolic activity of a non-phagocytic cell, the method comprising:
(a) contacting the cell with a culture medium so that the culture medium comprises the cell; and
(b) contacting the culture medium comprising the cell with a population of at least two nanoparticles of poly(ethyl acrylate-co-methyl methacrylate-co-trimethylammonioethyl methacrylate chloride) 1:2:0.1 for a period of time sufficient to increase the metabolic activity of the non-phagocytic cell, wherein, the average diameter of the nanoparticles of the population is from 55 nm to 100 nm.

16. The method according to claim 15, wherein the culture medium comprises a growth factor.

17. The method according to claim 15 or 16, wherein the culture medium comprises animal serum.

18. The method according to any of claims 15 to 17, wherein the cell is not contacted with a serum-free culture medium prior to step (b).

19. The method according to any of claims 15 to 18, wherein the population of at least two nanoparticles is a population of at least two naked nanoparticles.

20. The method according to any of claims 15 to 19, wherein the contacting of step (b) is for a period of time greater than 48 hours.

21. The method according to any of claims 15 to 19, wherein the contacting of step (b) is for a period of from 24 hours to 72 hours.

22. The method according to any of claims 15 to 21, further comprising measuring the metabolic activity of the non-phagocytic cell.

23. The method according to claim 22, wherein measuring the metabolic activity of the cell is performed using a tetrazolium dye other than MTT (3-(4,5-dimethylthiazol-2-yl)-2,5-diphenyltetrazolium bromide, a yellow tetrazole).

24. A method of extracting proteins from a serum sample, the method comprising:
(a) contacting a serum sample with a population of nanoparticles of poly(ethyl acrylateco-methyl methacrylate-co-trimethylammonioethyl methacrylate chloride) 1:2:0.1 for a period of time sufficient for the nanoparticles to bind to proteins present in the serum, to obtain a population of protein-bound nanoparticles; and
(b) isolating protein-bound nanoparticles of the population of protein-bound nanoparticles, to obtain a population of isolated protein-bound nanoparticles;
thereby extracting proteins from the serum sample.

25. The method according to claim 24, further comprising contacting a cell with isolated protein-bound nanoparticles of the population of isolated protein-bound nanoparticles.

26. The method according to claim 24 or 25, further comprising identifying a protein, or an Interpro domain of a protein, bound to an isolated protein-bound nanoparticle of the population of isolated protein-bound nanoparticles.

27. The method of claim 26, where identifying comprises the using mass spectrometry.

28. The method according to claim 26, comprising identifying an Interpro domain of a protein bound to an isolated protein-bound nanoparticle of the population of isolated protein-bound nanoparticles.

29. The method according to claim 28, wherein the identified Interpro domain is selected from: IPR006210:EGF-like, IPR000215:Protease_inhib_I4_serpin, IPR013783:Ig-like_fold, IPR016060:Complement_control_module, IPRO01254:Peptidase_S1_S6, IPRO01611:Leu-rich_rpt, IPR006209:EGF, IPR008985:ConA-likelec_gl, IPR000436:Sushi_SCR_CCP, IPR009003:Pept_cys/ser_Trypsin-like, IPR018039:Intermediate_filament_CS, IPRO11992:EF-hand-like_dom, PR002035:VWF_A, IPR001599:Macroglobln_a2, IPR000859:CUB, IPR008160:Collagen, IPR003961:Fibronectin_type3, IPRO04001:Actin_CS, IPR000264:Serumumin, IPR000001:Kringle, IPRO17857:Coagulation_fac_subgr_Gla_dom, IPR009053:Prefoldin, IPR018056:Kringle_CS, IPR000719:Prot_kinase_cat_dom, IPRO01791:Laminin_G, IPR000010:Prot_inh_cystat, IPR000884:Thrombospondin_1_rpt, IPRO12674:Calycin, IPR008979:Galactose-bd-like, IPR020837:Fibrinogen_CS, IPRO01304:C-typelectin, IPR002223:Prot_inh_Kunz-m, and IPRO17441:Protein_kinase_ATP_BS.

30. The method according to claim 26, comprising identifying a protein bound to an isolated protein-bound nanoparticle of the population of isolated protein-bound nanoparticles.

31. The method according to claim 30, wherein the identified protein is selected from: actin, gamma-enteric smooth muscle, kininogen-2 (isoform II), serpin A3-3, serpin A3-5, CD40 ligand, C-type lectin domain family 11 member A, talin-1, Ig heavy chain MemS-like, partial, SERPINA3-8, protein S100-A10 , mitochondrial fission 1 protein, factor XIIa inhibitor, sphingomyelin phosphodiesterase, pleckstrin, keratin, type II cytoskeletal 7, collectin-43, serpin A3-7, ras-related protein Rab-6B, proheparin-binding EGF-like growth factor, tRNA guanosine-2′-O-methyltransferase TRM13 homolog, asporin, biglycan, zinc finger and BTB domain-containing protein 48, nucleolar GTP-binding protein, aspartyl-tRNA synthetase, cytoplasmic solute carrier family 2_facilitated glucose transporter member 4, leucine-rich repeat transmembrane neuronal protein 4, toll-like receptor 6, v-src sarcoma (Schmidt-Ruppin A-2) viral oncogene homolog, coiled-coil domain-containing protein 147, transmembrane and TPR repeatcontaining protein 3, cylicin-1, GPI ethanolamine phosphate transferase 2, AFG3-like protein 2, dynamin-2, nebulette, structural maintenance of chromosomes protein 11, nardilysin, WD repeat-containing protein 17, tubulin polyglutamylase, TTLL5 isoform 1, citron Rho-interacting kinase, neurexin-2-beta, AT-rich interactive domain-containing protein 2, 5 carrying the IPR006210:EGF-like domain, LOC616876, LOC782051, LOC100139405, and C10orf18.

32. The method according to any of claims 24 to 31, wherein the serum sample is selected from: human serum, a human biological fluid, cerebrospinal fluid (CSF), urine, a transudate, an exudate, human cerebrospinal fluid (CSF), human urine, a human transudate, a human exudate, fetal bovine serum (FBS), bovine serum, chicken serum, newborn calf serum, rabbit serum, goat serum, horse serum, lamb serum, porcine serum, and a combination thereof.

33. A method for culturing a cell in vitro, the method comprising culturing the cell in a liquid culture composition comprising a protein/nanoparticle complex, wherein the protein/nanoparticle complex comprises:
a population of nanoparticles of poly(ethyl acrylate-co-methyl methacrylate-co-trimethylammonioethyl methacrylate chloride) 1:2:0.1, wherein the average diameter of the nanoparticles is from 30 nm to 100 nm, and wherein the population of nanoparticles comprises serum-derived proteins non-covalently bound to the nanoparticles.
